(19) United States
(12) Patent Application Publication
(10) Pub. No.: US 2022/0042040 A1
(43) Pub. Date: Feb. 10, 2022

(54) DUAL VECTOR SYSTEM FOR IMPROVED PRODUCTION OF PROTEINS IN ANIMAL CELLS

(71) Applicants: Universität für Bodenkultur Wien, Vienna (AT); ACIB GmbH, Graz (AT)

(72) Inventors: Reingard GRABHERR, Wien (AT); Wolfgang ERNST, Wien (AT); Krisztina KOCZKA, Wien (AT); Miriam KLAUSBERGER, Wien (AT)

(21) Appl. No.: 17/276,472

(22) PCT Filed: Sep. 13, 2019
(86) PCT No.: PCT/EP2019/074485

§ 371 Date: Mar. 15, 2021

(30) Foreign Application Priority Data
Sep. 17, 2018 (EP) 18194719.3

(51) Int. Cl. C12N 15/86

(52) U.S. Cl. CPC C12N 15/86; C12N 2710/14143; C12N 2710/14121

(57) ABSTRACT

The present invention refers to a dual vector system for production of one or more recombinant proteins in cells of insect origin comprising a first viral vector comprising a T7 promoter operably linked to a targeting sequence comprising a non-coding sequence and which expression can suppress one or more proteins essential for virus production, followed by a T7 termination sequence, and a second viral vector comprising a promoter operably linked to a T7 RNA polymerase encoding sequence, and at least one gene sequence located on the first and/or second vector encoding one or more recombinant proteins of interest. The invention further refers to recombinant insect cells comprising the dual vector system and methods for producing recombinant proteins using said dual vector CA system.

### FIELD OF THE INVENTION

The present invention refers to a dual vector system for production of one or more recombinant proteins in cells of insect origin comprising a first baculovirus vector comprising a T7 promoter operably linked to a targeting sequence comprising a non-coding sequence and which expression can suppress one or more proteins essential for virus production, followed by a T7 termination sequence, and a second baculovirus vector comprising a promoter operably linked to a T7 RNA polymerase encoding sequence, and at least one gene sequence located on the first and/or second vector encoding one or more recombinant proteins of interest. The invention further refers to recombinant insect cells comprising the dual vector system and methods for producing recombinant proteins using said dual vector system.

### BACKGROUND OF THE INVENTION

Insect and mammalian cells are among the most important animal cells used for recombinant protein expression in biotechnology.

Applications of insect cells include not only the production of various viral capsid and envelope proteins for use as vaccines or for analytical purposes, but also many enzymes, membrane proteins and self-assembled nanoparticles have been successfully produced in Spodoptera frugiperda (Sf9) and Trichoplusia ni (HighFive) cells. Insect cells are feasible for the expression of intracellular proteins as well as secreted, often complex and glycosylated proteins. A commonly used recombinant protein expression system is the baculovirus expression vector system (BEVS) using insect cells. Baculoviruses have very species-specific tropisms among invertebrate cells and are not known to replicate in mammalian or other vertebrate animal cells, therefore the baculovirus expression system has become one of the most widely used eukaryotic systems for production of recombinant proteins. The baculovirus Autographa californica multicapsid nuclear polyhedrosis virus (AcMNPV) is by far the most common vehicle in this system.

Baculoviruses are a family of large, rod-shaped, enveloped viruses that contain circular double-stranded DNA genomes ranging from 80-180 kilo base pairs (kbp). Baculovirus infection of host cells can be divided into three distinct phases: “early” (0-6 h post-infection (p.i.)), “late” (6-24 h p.i.) and “very late” (18-24 to 72 h p.i.). The baculovirus “very late” promoters display extremely high rates of transcription relative to host cell promoters and early or late baculovirus promoters. Therefore, the basic idea behind prior-art baculovirus expression of recombinant proteins in insect cells was that the DNA sequence coding for a recombinant protein of interest is shuttled into the baculovirus genome under the control of such a “very late” promoter to express high levels of the recombinant protein during (very late stages of) infection of the cultured host cell.

A number of technological improvements have eliminated the original tedious procedures required to create and culture recombinant baculoviruses. Baculovirus expression systems allow for recombinant genes to be shuttled into the baculovirus genome through recombination of baculovirus DNA and recombinant gene-containing plasmids in cells, or in vitro. Using any of these methods, sufficient recombinant protein-encoding viruses are typically generated to infect insect cell culture volumes in the order of a few milliliters. However, for baculovirus-based applications such as vaccine production, hundreds, even thousands of liters of insect cell culture are required to generate the desired levels of recombinant protein. Large scale baculovirus expression requires an exponential increase in the number of recombinant viruses from the starting point of small numbers of recombinant viruses to millions of virus particles. This requires intact virus replication as well as production of infectious virions to spread the infection. Scale-up is typically carried out by allowing the recombinant viruses to replicate in sequentially larger culture volumes and harvesting the resultant virus particles for generation of a master virus seed stock. Subsequent recombinant protein production using this virus seed stock is then typically carried out in a separate culture setup. During the scale-up phase, the generation of replication competent, infectious baculovirus particles is required. However, during protein production phase, co-production of new infectious virions to spread the infection throughout the culture used for production of recombinant protein is not desired.

The co-production of baculovirus virions together with the recombinant protein of interest indeed can represent a serious drawback of baculovirus expression systems, as it can be difficult and costly to separate the co-produced baculovirus from the recombinant protein.

In prior art baculovirus expression systems, recombinant proteins are constitutively transcribed and translated at very high levels and therefore require a significant fraction of the cell's total metabolic capacity. In addition, recombinant proteins are not essential for replication of the virus. This results in a high selection pressure for generating mutant viruses, which do not transcribe or translate the recombinant protein, such as those that no longer carry the non-essential recombinant protein expression cassette. It is expected that scaling up baculovirus expression to larger culture volumes will result in a progressive accumulation of viruses that do not carry a functional recombinant protein expression cassette (Pijlman et al., 2003). This is not too surprising, given the well documented phenomenon of plasmid loss experiments, which demonstrate that non-essential DNAs are very rapidly removed from a wide-range of cell types in the absence of selection markers to maintain them (Boe, 1996).

A further strategy to overcome the drawbacks of co-expression of baculovirus virion particles and recombinant proteins is the use of helper cell lines. Thereby, a gene that is essential for virus budding is deleted from the baculovirus genome and a helper cell line providing the missing gene is generated, that allows propagation of this virus. However, these helper cell lines suffer from instability and poor virus production (Marek et al., 2011).

Lee H. S. et al. (2015) report the use of siRNA targeting glycoprotein 64 or single-stranded DNA-binding protein to inhibit baculovirus replication during overexpression of recombinant foreign proteins.

Van Poeljwik F. et al. (1995) describe a hybrid recombinant baculovirus-bacteriophage T7 expression system for transient expression of plasmids with foreign genes in insect cells.

In Steele K. H. et al. (2017) insect cells were transformed with an expression plasmid harboring vankyrin gene encoding an anti-apoptotic protein.

There is an unmet demand for an improved or alternative method for the production of a recombinant protein with reduced baculovirus virion contamination, where both the scale up and recombinant protein production phases are carried out in the same cell line. Hence, it is an objective of the invention to provide a method for the production of a recombinant protein using a baculovirus system in insect or mammalian cells, wherein the production of baculovirus virions is provided during baculovirus amplification processes, but suppressed during recombinant protein production phase.

### SUMMARY OF THE INVENTION

The object is solved by the subject matter of the invention.

The dual vector system of the present invention provides a variable expression system in animal cells such as insect or mammalian cells without the need of helper cell lines. Said system allows for down-regulation of essential genes of the baculovirus genome. The method for producing recombinant protein using the dual vector system of the invention is broadly applicable and allows for producing high yields of recombinant proteins even in large industrial-scale insect and mammalian cell cultures.

By virtue of the method of the invention, it is possible to produce recombinant protein in animal cells, specifically in insect or mammalian cells with reduced or no contamination of baculovirus virions, and to passage viruses expressing recombinant protein relative to what is achieved by currently available methods.

According to the invention there is provided a dual vector system for production of one or more recombinant proteins in animal cells, specifically of insect or mammalian origin comprising
a first viral vector comprising a T7 promoter operably linked to a targeting sequence comprising a non-coding sequence and which expression can suppress one or more proteins essential for virus production, followed by a T7 termination sequence or derivatives thereof, and
a second viral vector comprising a promoter operably linked to a T7 RNA polymerase-encoding sequence, and
at least one gene sequence located on the first and/or second viral vector encoding one or more recombinant proteins of interest.

According to a specific embodiment, the vectors are baculovirus vectors.

According to an embodiment of the invention there is provided a dual vector system for production of one or more recombinant proteins in insect cells comprising
a first baculovirus vector comprising a T7 promoter operably linked to a targeting sequence comprising a non-coding sequence and which expression can suppress one or more proteins essential for virus production, followed by a T7 termination sequence or derivatives thereof, and
a second baculovirus vector comprising a promoter operably linked to a T7 RNA polymerase-encoding sequence, and
at least one gene sequence located on the first and/or second viral vector encoding one or more recombinant proteins of interest.

The bacterial T7 system allows universal use in all insect and mammalian cells thus no cell specific polymerase such as polymerase III is required.

According to an embodiment, the T7 RNA polymerase is linked to a nucleus localization sequence (NLS).

Generally, the dual vector system allows suppression of baculovirus virion production during the recombinant protein production phase. By the vector and methods of the invention, expression of a gene product essential for baculovirus virion production can be suppressed, thereby inhibiting virion assembly following virus amplification process. Thereby time and effort for recombinant protein purification can be reduced after cell harvest.

According to an embodiment, the expression product of the targeting sequence is a non-coding (nc) RNA selected from the group of guide RNAs (gRNA), endogenous primary-micro RNAs (pri-miRNA), artificial micro RNAs (amiRNA), small interfering RNAs (siRNA), short hairpin RNAs (shRNA), long hairpin RNAs (IhRNA), and polycystronic shRNAs.

According to an embodiment of the invention, the ncRNA encoded by the targeting sequence employs a hairpin structure which is further processed by one or more cellular enzymes, specifically Dicer and/or Drosha.

According to a specific embodiment, two, three or more gene product(s) or proteins essential for baculovirus virion assembly can be targeted. The two, three or more gene products essential for baculovirus virion assembly may be targeted by one ncRNA or, as an alternative, by several ncRNAs encoded by one, two, three or more viral vectors. As an example, gp64 and vp80 can be targeted simultaneously, thereby resulting in gradual or complete inhibition of protein expression.

In a further embodiment of the invention, the first viral vector further comprises a gene sequence encoding a Cas9 nuclease or other RNA-programmable nucleases such as Cpf1 nucleases optionally linked to one or two NLS.

According to a further embodiment, the second vector further comprises a gene sequence encoding a Cas9 nuclease or other RNA-programmable nucleases such as Cpf1 nucleases, optionally linked to a NLS.

By antisense RNA or CRISPR/Cas9 technology, genes that are essential for baculovirus budding are being downregulated during the time of recombinant protein production thereby reducing or avoiding baculovirus particles to be present in the final product.

In yet a further embodiment, the target gene sequence suppresses or functionally knocks out a gene product essential for infectious baculovirus virion generation, specifically selected from the group consisting of vp80, vp39, vp1054, gp64, p74, p24, vp1054, lef-1, lef-2, lef-4, lef-9, lef-11, pk1, vlf-1, bv-c42, bv-c27, Ac9, Ac25, Ac51, Ac53, Ac73, Ac75, Ac76, Ac78, Ac79, Ac81, Ac81, Ac82, Ac83, Ac92, Ac106/107, Ac109, Ac132, Ac146, 38K, and p6.9.

In a further embodiment, the promoter linked to the T7 polymerase is an early viral promoter, specifically selected from iE1, pe38, me53, lef3, gp64, vp39, and he65 promoter or a cellular promoter.

According to a specific embodiment, the viral vector is derived from nuclear polyhedrosis virus (NPV), specifically from Autographa californica multiple nuclear polyhedrosis virus (AcMNPV).

Herein provided is further a recombinant insect or mammalian cell line comprising the dual vector system of the invention.

Specifically, the cell line is derived from Spodoptera frugiperda, Trichoplusia ni, Bombyx mori, Plutella sylostella, Manduca sexta and Mamestra brassicae, Helicoverpa armigera, Antheraea pernyi, Culex nigripalpus, Drosophila melanogaster, specifically selected from the group of Spodoptera frugiperda and Trichoplusia ni cells/cell lines, specifically from the group of Sf9, Sf21, Mimic™ Sf9 and Tnms42, Tnao38, High Five™, and cells/cell lines, respectively.

Further provided according to the invention is a method for production of a recombinant protein in cells of animal origin, specifically of insect or mammalian origin, comprising the steps of:
introducing the dual vector system described herein into animal, specifically insect or mammalian cells,
cultivating the cells under conditions allowing expression of the recombinant protein and simultaneous downregulation of expression of the target protein, and
isolating the recombinant protein.
Specifically, the method described above is performed with insect cells and baculovirus vector.

Further provided is a method for production of a recombinant protein in cells of animal origin, specifically of insect or mammalian origin, comprising the steps of:
introducing the first viral vector of the dual vector system as described herein comprising a gene sequence encoding a recombinant protein into animal cells, specifically insect or mammalian cells,
cultivating said cells under conditions allowing virus propagation and expression of the recombinant protein,
introducing the second viral vector of the dual vector system as described herein into said cells,
cultivating the cells under conditions wherein expression of the target protein is downregulated, and
isolating the recombinant protein.

In an embodiment of the invention, expression of the target protein is downregulated due to the presence of effective amounts of ncRNA effector specifically targeting mRNA encoding said target protein.

According to a further embodiment, expression of the target protein is downregulated due to functional knock-out of the target gene sequence by RNA-programmable nucleases

### FIGURES

FIG. 1: Plasmid-based screening of the amiRNA constructs with flow cytometry

FIG. 2: In vitro transcription assay for the verification of the recombinant T7 RNAP's functionality

FIG. 3: Mechanism of the inducible silencing system targeting eYFP

FIG. 4: Flow cytometry results of the virus-based inducible system

FIG. 5: RT-qPCR results

FIG. 6: Schematic of the AcCRISP-AcT7 dual vector system

FIG. 7: Schematic of DNA templates employed for T7 RNAP-mediated in vitro gRNA synthesis

FIG. 8: Functionality of T7 RNAP-transcribed gRNAs in Cas9 In Vitro Assays

FIG. 9: Sequence data

FIG. 10: Schematic of sgRNA transcription cassette for in vivo sgRNA transcription.

FIG. 11: Schematic of T7RNAP expression cassette

FIG. 12: Schematic of viral constructs

FIG. 13: gp64-Knock-down effect as evaluated by qPCR

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Janeway et al, “Immunobiology” (5th Ed., or more recent editions), Garland Science, New York, 2001. The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

Singular and plural forms can be used interchangeably herein if not otherwise indicated.

As used herein, the term “vector” refers to a nucleic acid molecule capable of transporting another nucleic acid to which it has been linked.

The term “viral vector” refers to a nucleic acid molecule capable of transporting another nucleic acid to which it has been linked. Viral vectors comprise additional DNA segments ligated into the viral genome. Vectors are capable of autonomous replication in a host cell into which they are introduced. Specifically, the system as described herein comprises at least one viral vector containing a T7 promoter operably linked to a targeting sequence, which is a non-coding sequence and which expression can suppress one or more proteins essential for virus production, followed by a T7 termination sequence and optionally containing one or more sequences encoding recombinant proteins of interest. Herein, also two, three or more vectors each containing different targeting sequences can be comprised in a host cell. The system further comprises at least one further viral vector comprising a promoter operably linked to a T7 RNA polymerase encoding sequence, and optionally further containing sequences encoding one or more recombinant proteins of interest. Herein, also two, three or more vectors each containing different targeting sequences can be comprised in a host cell.

Specifically, the viral vector is a “baculovirus vector” which is a covalently closed circular double stranded polynucleotide, which can accommodate large fragments of foreign DNA. Baculovirus vectors are based upon Baculoviridae viruses, which infect arthropods as their natural hosts. The family Baculoviridae comprises the genera alphabaculovirus, betabaculovirus, comprising Cydia pomonella granulovirus, deltabaculovirus, comprising Culex nigripalpus nucleopolyhedrovirus and gammabaculovirus, comprising Neodiprion lecontei nucleopolyhedrovirus.

Baculovirus vectors mainly used as expression vectors are based upon Autographa californica multiple nucleopolyhedrovirus (AcMNPV), an alphabaculovirus, which was isolated from alfalfa looper larvae. Examples of commercially available baculovirus vectors which can be used herein include the BacMam system, the Baculovirus Expression Vector System (from BD Biosciences, San Diego Calif. USA), Baculovirus Expression System—BacPAK (from Clontech Laboratories, Inc., Mountain View Calif. USA), pFastBac vector (Invitrogen) and BD BaculoGold (BD Biosciences), flashBAC™ (Oxford Expression Technologies EP), BacVector® 1000/2000/3000 (Novagen®), BAC-TO-BAC® (Invitrogen™), BaculoDirect™ (Invitrogen™) and MultiBac™, VLPFactory™, SynBac™, KinaseFactory™, Hormone Receptor Factory™, DeGlyco Factpry™, ComplexLINK™, SweetBac™ (Geneva Biotech). These baculovirus-based insect cell expression systems are essentially based on expressing recombinant proteins by placing them under the control of very late baculovirus promoters, namely the polh and/or p10 promoters. Any of these baculovirus expression systems could be conveniently modified to comply with the present invention by incorporating controllable transcriptional activators or repressors into the system as described herein, thereby directly or indirectly controlling recombinant protein expression. Accordingly, the baculovirus expression systems of the present invention can be based on any one of the commercially or academically available baculovirus expression systems.

For baculovirus vectors and baculovirus DNA, as well as insect cell culture procedures, see, for example, O'Reilly et al., Baculovirus Expression Vectors: A Laboratory Manual, Oxford University Press, New York, 1994. The baculovirus vector may contain additional elements, such as an origin of replication, one or more selectable markers allowing amplification in the alternative hosts, such as mammalian and insect cells. In certain embodiments, there are provided baculovirus vectors that contain cis-acting control regions effective for expression in a host operatively linked to the polynucleotide to be expressed. Appropriate trans-acting factors are either supplied by the host, supplied by a complementing vector, or supplied by the vector itself upon introduction into the host.

The viral vectors can be introduced into the host cell by any of a number of appropriate means, including infection (where the vector is an infectious agent, such as a viral or baculovirus genome), transduction, transfection, transformation, electroporation, microprojectile bombardment, lipofection, or any combinations thereof. A preferred method of genetic transformation of the host cells is infection.

Each of the vectors may be introduced alone or with other vectors of different type. The vectors may be introduced independently, co-introduced or introduced joined to other vectors using standard techniques for co-transfection and selection.

In another embodiment, more than one viral vector containing genes encoding different recombinant proteins of interest are introduced into the host cell.

The recombinant proteins to be produced using the dual vector system described herein can be any protein of interest.

The term “recombinant protein of interest (RPOI)” or protein of interest (POI) as used herein refers to a polypeptide or a protein that is produced by means of recombinant technology in a host cell. More specifically, the protein may either be a polypeptide not naturally occurring in the host cell, i.e. a heterologous protein, or may be native to the host cell, i.e. a homologous protein to the host cell, but is produced, for example, by transformation, infection, transfection, transduction with a self-replicating vector containing the nucleic acid sequence encoding the RPOI, or upon integration by recombinant techniques of one or more copies of the nucleic acid sequence encoding the RPOI into the genome of the host cell, or by recombinant modification of one or more regulatory sequences controlling the expression of the gene encoding the RPOI, e.g. of the promoter sequence.

Further, the DNA sequence encoding the recombinant protein can be a naturally existing DNA sequence or a non-natural DNA sequence. The recombinant protein can be modified in any way. Non-limiting examples for modifications can be insertion or deletion of post-translational modification sites, insertion or deletion of targeting signals, fusion to tags, proteins or protein fragments facilitating purification or detection, mutations affecting changes in stability or changes in solubility or any other modification known in the art. In certain embodiments of the invention the recombinant protein is a biopharmaceutical product, which can be any protein suitable for therapeutic or prophylactic purposes in mammals. Examples for proteins that can be produced by the method of the invention are, without limitation, enzymes, regulatory proteins, receptors, peptides, e.g. peptide hormones, cytokines, membrane or transport proteins. The proteins of interest may also be antigens as used for vaccination, vaccines, antigen-binding proteins, immune stimulatory proteins, allergens, full-length antibodies or antibody fragments or derivatives. Antibody derivatives may be selected from the group of single chain antibodies, (scF), Fab fragments, Fr fragments, single domain antibodies (VH or VL, fragment) or domain antibodies (nanobodies). In some specific embodiments of the invention, the recombinant protein is an enveloped or non-enveloped virus-like particle or nanoparticle.

The term “heterologous” with respect to a nucleotide or amino acid sequence or protein, refers to a compound which is either foreign, i.e. “exogenous”, such as not found in nature, to a given host cell; or that is naturally found in a given host cell, e.g., is “endogenous”, however, in the context of a heterologous construct, e.g., employing a heterologous nucleic acid, thus “not naturally-occurring”. The heterologous nucleotide sequence as found endogenously may also be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature (i.e., “not natively associated”). Any recombinant or artificial nucleotide sequence is understood to be heterologous. An example of a heterologous polynucleotide or nucleic acid molecule comprises a nucleotide sequence not natively associated with a promoter, e.g., to obtain a hybrid promoter, or operably linked to a coding sequence, as described herein. As a result, a hybrid or chimeric polynucleotide may be obtained. A further example of a heterologous compound is a RPOI encoding polynucleotide or gene operably linked to a transcriptional control element, e.g., a promoter, to which an endogenous, naturally-occurring POI coding sequence is not normally operably linked.

The term “operably linked” as used herein refers to the association of nucleotide sequences on a single nucleic acid molecule, i.e. the vector, in a way such that the function of one or more nucleotide sequences is affected by at least one other nucleotide sequence present on said nucleic acid molecule. For example, a promoter is operably linked with a coding sequence of a recombinant gene or with the targeting sequence, when it is capable of effecting the expression of that coding or targeting sequence. Specifically, such nucleic acids operably linked to each other may be immediately linked, e.g. without further elements or nucleic acid sequences in between the nucleic acid encoding the signal peptide and the nucleic acid sequence encoding a targeting sequence.

Animal cells or cells of animal origin can be any host cell from standard or conventional cell lines known in the art. Animal cells are the basic unit of life in organisms of the kingdom Animalia. They are eukaryotic cells, meaning that they have a true nucleus and specialized structures called organelles that carry out different functions. According to the inventions, insect, avian and mammalian cells are preferred.

Insect cells as encompassed herein are any host cells from a standard or conventional insect cell line known in the art, such as, but not limited to cell lines derived or originating from Spodoptera frugiperda, Trichoplusia ni, Bombyx mori, Plutella sylostella, Manduca sexta and Mamestra brassicae, Helicoverpa armigera, Antheraea pernyi, Culex nigripalpus, Heliothis virescens, Heliothis zea, Mamestra brassicas, Estigmene acrea and Drosophila melanogaster, specifically Sf9, Sf21, High Five™ ((BT1-TN-5B1-4; insect cell line that originated from the ovarian cells of Trichoplusia ni), Mimic™ Sf9, BT1-Ea88, Tnms42, Tnao38, Tn-368, mb0507, Tn mg-1, and Tn Ap2, among other cells.

Mimic™ Sf9 cell line is a derivative of the Sf9 insect cell line. Cells are modified to stably express a variety of mammalian glycosyltransferases. Typically, insect cells are unable to process N-glycans to the extent that mammalian cells do. This can affect protein structure, function, antigenicity, and enzymatic activity. The addition of mammalian glycosyltransferases to the Mimic™ Sf9 Insect Cells allows for production of biantennary, terminally sialyated N-glycans from insect cells. The cells can be used to produce more mammalian-like proteins in baculovirus and stable insect expression systems.

Mammalian cells useful for the method described herein are capable of expressing recombinant proteins and are well known in the art. These cell or cell lines can be, but are not limited to CHO, COS, Vero, Hela, BHK, HEK293, Hek293T, Hek293S, Hek293FT, 3T3, WI 38, BT483, HTB2, BT20, T47D, NSO, HKB-11, MEF and Sp-2 cell lines.

The term “promoter” as used herein is a region of DNA that facilitates the transcription of a particular gene. It is an expression control element that permits binding of RNA polymerase and the initiation of transcription. Promoters are typically located adjacent to the genes they regulate, on the same strand and upstream (towards the 5′ region of the sense strand).

The term “T7 promoter” corresponds to the promoter region of the bacteriophage T7 or to functional analogues or derivatives thereof, which promoter is capable of initiating transcription of a targeting sequence as described herein downstream thereto. Thus, the T7 promoter is operably linked to the targeting sequence. Specifically, the promoter is a T7 promoter selected from T7_{A1}, T7_{A2}, T7_{A3}.

Specifically, the T7 promoter comprises the sequence TAATACGACTCACTATA, SEQ ID No. 18.

The promoter operably linked to the T7 RNA polymerase can be any inducible or constitutive promoter that is recognized by an RNAP encoded by an RNAP gene comprised in the chromosome of the host. Exemplarily, the promoter can be an early viral promoter such as but not limited to iE1, pe38, me53, lef3, gp64, vp39, and he65 promoter or a cellular promoter.

The term “T7 termination sequence” refers to any sequence which stops elongating T7 RNA polymerase. Examples for T7 termination sequences include but are not limited to Tphi terminator, a late terminator found in the T7 genome, rrnBT1 terminator of E. coli (Jeng et al., 1992).

The term “T7 RNA polymerase” refers to the RNA polymerase from T7 bacteriophage, specifically encoded by the T7 gene 1.

The term “one or more proteins essential for virus production” refers to a protein or polypeptide, which upon inactivation or deletion results in a baculovirus phenotype with suppressed or reduced numbers of baculovirus virions, including budded virions and occlusion-derived virions. These may include capsid proteins or proteins required at any step of capsid assembly or for transport of the virions out of the cell. Such a protein may be identified by deletion of the encoding gene and analyzing the baculovirus phenotype as known in the art. Additionally RNAi technology as described herein may be used to silence expression of said gene and analyzing the baculovirus phenotype as known in the art. Such essential proteins can be, but are not limited to vp80 baculovirus capsid protein, vp39, vp1054, gp64, p74, p24, vp1054, lef-1, lef-2, lef-4, lef-9, lef-11, pk1, vlf-1, bv-c42, bv-c27, Ac9, Ac25, Ac51, Ac53, Ac73, Ac75, Ac76, Ac78, Ac79, Ac81, Ac81, Ac82, Ac83, Ac92, Ac106/107, Ac109, Ac132, Ac146, 38K, and p6.9.

The terms “express” and “expression” as used herein shall mean allowing or causing the information in a gene, RNA or DNA sequence to become manifest, for example, producing a protein by activating the cellular functions involved in transcription and translation of a corresponding gene. Nucleic acid molecules containing a desired coding sequence of an expression product such as e.g., a recombinant protein as described herein, and control sequences such as e.g., a promoter in operable linkage, may be used for expression purposes. A DNA sequence is expressed in or by a cell to form an “expression product” such as an RNA (e.g., mRNA) or a protein. The expression product itself may also be said to be “expressed” by the cell. Hosts transformed or transfected with these sequences are capable of producing the encoded proteins.

The term “suppress” or “suppressing” as used herein refers to any interference of protein expression or baculovirus virion production, resulting in reduced expression levels of said protein or reduced amounts of baculovirus virions compared to the respective wild-type baculovirus expression systems. This includes repressed protein expression due to the presence of effective amounts of targeting sequence comprising a non-coding sequence such as ncRNA selected from the group of guide RNAs (gRNA), endogenous primary-micro RNAs (pri-miRNA), artificial micro RNAs (amiRNA), small interfering RNAs (siRNA), short hairpin RNAs (shRNA), long hairpin RNAs (IhRNA), and polycystronic shRNAs specifically targeting mRNA encoding said protein, wherein effective amounts means that expression levels of said protein are substantially reduced, i.e., by at least 20, 30, 40, 50, 60, 70, 75, 80, 85, 90, 95, 99 or 100% compared to the respective wild-type baculovirus expression system. Said RNAs specifically are double-stranded. The respective non coding RNAs can be of any length appropriate for the method described herein which can be determined by the skilled person according to methods known in the art. Specifically the RNAs may be of about 10 to 50 nt length, specifically of about 15 to 40 nt, specifically about 20 to 30, more specifically about 25-30 nt.

The term “gRNA” as used herein refers to the guide RNA (gRNA or sgRNA) which is a short synthetic RNA composed of a scaffold sequence necessary for Cas9 or Cpf1-binding and a user-defined spacer that defines the target to be modified. Herein the target refers to any sequence encoding a gene product essential for virus production, i.e. baculovirus virion assembly. Cas9 or Cpf1 will then only cleave a locus on the respective sequence if the gRNA spacer sequence shares sufficient homology with the target DNA.

This also includes repressed and knocked out protein expression or genome editing due to the presence of effective amounts of Cas9 nuclease, specifically leading to functional knock out of the respective protein. Delivery of the CRISPR/Cas9 components—the Cas9 nuclease and gRNA—may be either in the format of (1) DNA encoding for the two components, (2) mRNA for Cas9 translation together with a separate gRNA or (3) a ribonucleoprotein complex consisting of recombinantly expressed Cas9 in complex with the gRNA (Newman M. and Ausubel FM., 2016, Curr Protoc Mol Biol 115, 31.41-31.4.6), the method of (1) being preferred herein. In vivo, the Cas9:gRNA complex binds to the DNA sequence to be modified and the Cas9 nuclease introduces a double-strand-break at the specified gene or locus of interest. Highly conserved non-homologous end joining (NHEJ) DNA repair mechanisms repair the double-strand break in vivo, thereby creating stall insertions or deletions (indels). This ultimately results in frame-shift mutations and destroys the open-reading frame of the gene. Alternatively, when a DNA template with a desired mutation is supplied in vivo this may result in the substitution of the desired sequences at the site of the DSB by homology-directed repair (HDR) mechanisms (Hsu et al., 2014).

In alternative embodiments, one ncRNA targets one gene product essential for baculovirus virion assembly, wherein the ncRNA can form multiple siRNAs specifically targeting different target sequences of the same open reading frame coding for a gene product essential for baculovirus virion assembly. RNA interference is a conserved biological process using short RNA molecules, the small interfering RNAs (siRNAs), to sequence-specifically destroy target mRNAs and thus regulate gene expression (Hannon, 2002). SiRNAs are about 20-25-nucleotide (nt) long non-coding double-stranded RNA molecules that are produced in response to foreign nucleic acid originating from exogenous invaders, such as viruses or transposons (Dana et al., 2017). One way of taking advantage of the RNAi pathway and to target selected genes for silencing is to introduce synthetic siRNAs in vitro by embedding their sequences in endogenous primary-microRNA (primiRNA) transcripts that serve as backbones (Haley et al., 2008; Zhang et al., 2014). This way the primiRNA transcript carrying the synthetic siRNA, or so called artificial microRNA (amiRNA), is recognized by the host cell's microRNA-processing pathway and it is digested in vivo by a class 2 ribonuclease III enzyme, e.g. Drosha and an endoribonuclease or helicase with RNAse motif, e.g. Dicer to produce the precursor miRNA and the mature amiRNA, respectively (Bofill-De Ros and Gu, 2016). Specifically, the pri-miRNA transcript of Autographa californica multiple nucleopolyhedrovirus miR-1 can be used as a backbone to harbour an amiRNA targeting the protein essential for virion assembly.

Specifically, the artificial miRNAs can comprise the sequences of SEQ ID Nos. 1 to 9. In another embodiment one ncRNA targets two or more gene products essential for baculovirus virion assembly, e.g., two or three gene products essential for baculovirus virion assembly.

In a specific embodiment, the gene product essential for infectious baculovirus virion generation can be, but is not limited to vp80, vp39, vp1054, gp64, p74, p24, vp1054, lef-1, lef-2, lef-4, lef-9, lef-11, pk1, vlf-1, bv-c42, bv-c27, Ac9, Ac25, Ac51, Ac53, Ac73, Ac75, Ac76, Ac78, Ac79, Ac81, Ac81, Ac82, Ac83, Ac92, Ac106/107, Ac109, Ac132, Ac146, 38K, and p6.9.

The term “gene” as used herein refers to a DNA sequence that comprises at least promoter DNA, optionally including operator DNA, and coding DNA which encodes a particular amino acid sequence for a particular peptide, polypeptide or protein. Promoter DNA is a DNA sequence which initiates, regulates, or otherwise mediates or controls the expression of the coding DNA. Promoter DNA and coding DNA may be from the same gene or from different genes, and may be from the same or different organisms.

The term “recombinant” as used herein shall mean “being prepared by or being the result of genetic engineering”. A recombinant host specifically comprises a recombinant expression vector or cloning vector, or it has been genetically engineered to contain a recombinant nucleic acid sequence, in particular employing nucleotide sequence foreign to the host. A recombinant protein is produced by expressing a respective recombinant nucleic acid in a host.

The term “nuclear localization signal” or “NLS” as used herein refers to an amino acid sequence, or a nucleotide sequence encoding such AA sequence, that tags a protein for import into the cell nucleus, specifically by nuclear transport. Typically, this signal consists of one or more short sequences, preferably of positively charged lysines or arginines exposed on the protein surface. The amino acid sequence of an NLS specifically comprises about 4, 5, 6, 7, 8, 9, 10, 11, 12, 13, 14, 15, 16, 17, 18, 19 or 20 or more amino acids.

The role of specific amino acid sequences in targeting proteins to the cell nucleus has already been demonstrated in higher eukaryotes. Although an absolute consensus NLS has not yet been defined, a number of NLS have been studied in some detail. Typically, this signal consists of one or more short sequences of positively charged lysines or arginines exposed on the protein surface. The NLS most widely studied is a seven-amino-acid oligopeptide (Pro-Lys-Lys-128-Lys-Arg-Lys-Val, SEQ ID NO. 29) (the position of the second lysine in the simian virus 40 (SV40) T antigen is denoted) that is both necessary and sufficient to direct SV 40 large T antigen to the nucleus in mammalian cells. This oligopeptide is able to direct a number of otherwise cytoplasmic proteins to the nucleus when fused to these proteins either by genetic engineering followed by expression in vivo or by chemical cross-linking in vitro. Another well described NLS comprises the nucleoplasmin nuclear localisation signal (Lys-Arg-Pro-Ala-Ala-Thr-Lys-Lys-Ala-Gly-Gln-Ala-Lys-Lys-Lys-Lys, (SEQ ID NO. 19).

In a specific embodiment the T7 RNA polymerase and/or the Cas9 nuclease sequence are linked to “nucleus localization sequences”, “NLS”, which direct the RNA polymerase or the Cas9 nuclease to the nucleus by nuclear transport. In a specific embodiment, two or more NLS sequences can be used for targeting a sequence to the nucleus. Specifically, the NLS is SV40 T antigen nuclear location signal, specifically having the coding sequence CCTAAGAAGAAGAGAAAAGTC (SEQ ID No. 24), or is the nucleoplasmin nuclear localisation signal having the coding sequence AAAAGGCCGGCGGCCACGAAAAAGGCCGGCCAGGCAAAAAAGAAAAAG (SEQ ID No. 27) or a functional fragment or derivative thereof with at least 80%, specifically at least 90%, more specifically at least 95% sequence identity. Specifically, the NLS signal is located at the N-terminus of the T7 RNA polymerase. The SV40 NLS is located at the N-terminus of the Cas9 nuclease and the nucleoplasmin NLS is located at its C-terminus.

As described herein, a method for producing recombinant proteins in cells and cell lines of insect or mammalian origin is provided. The dual vector system described herein is thereby introduced into the mammalian or insect cells, i.e. by treating the cells with any method known to the skilled person in the art to bring the DNA encoding the viral vectors, specifically encoding the baculovirus expression system or the baculovirus comprising the DNA encoding the baculovirus expression system into an insect or mammalian cell. This comprises methods such as transfection, microinjection, transduction and infection. Methods for transfecting DNA into insect cells are known to the person skilled in the art and can be carried out, e.g., using calcium phosphate or dextran, by electroporation, nucleofection or by lipofection. The cells are then cultivated under conditions well known in the art allowing expression of the recombinant protein and simultaneous downregulation of expression of the target protein, and the recombinant protein is isolated by any purification methods applicable and known in the art.

The vectors of the dual system described herein can be simultaneously introduced into the cells. As an alternative the first viral vector comprising a gene sequence encoding a recombinant protein is introduced into the insect or mammalian cells, said cells are cultivated under conditions allowing simultaneous virus propagation and expression of the recombinant protein. After an appropriate time period determined by the skilled person, the second viral vector of the dual vector system is introduced into the cells. The cells are further cultivated under conditions wherein expression of the target protein which is essential for virus propagation is downregulated, and the recombinant protein is isolated and optionally purified.

The invention further provides following items:

1. Dual vector system for production of one or more recombinant proteins in cells of animal origin, specifically of insect or mammalian origin, comprising
a first viral vector comprising a T7 promoter operably linked to a targeting sequence comprising a non-coding sequence and which expression can suppress one or more proteins essential for virus production, followed by a T7 termination sequence, and/or
a second viral vector comprising a promoter operably linked to a T7 RNA polymerase encoding sequence, and
at least one gene sequence located on the first and/or second vector encoding one or more recombinant proteins of interest.

2. The dual vector system of item 1, wherein the viral vector is a baculovirus vector.

3. Dual vector system for production of one or more recombinant proteins in cells of insect origin, comprising
a first baculovirus vector comprising a T7 promoter operably linked to a targeting sequence comprising a non-coding sequence and which expression can suppress one or more proteins essential for virus production, followed by a T7 termination sequence, and
a second baculovirus vector comprising a promoter operably linked to a T7 RNA polymerase encoding sequence, and
at least one gene sequence located on the first and/or second vector encoding one or more recombinant proteins of interest.

4. The dual vector system of any one of items 1 to 3, wherein the T7 RNA polymerase is linked to a nucleus localization sequence (NLS).

5. The dual vector system according to item 1 to 4, wherein the expression product of the targeting sequence is an non-coding (nc)RNAi selected from the group of guide RNAs (gRNA), endogenous primary-micro RNAs (pri-miRNA), artificial micro RNAs (amiRNA), small interfering RNAs (siRNA), short hairpin RNAs (shRNA), long hairpin RNAs (IhRNA), polycystronic shRNAs.

6. The dual vector system according to any one of items 1 to 5, wherein the ncRNA encoded by the targeting sequence is further processed by one or more cellular enzymes, specifically Dicer and/or Drosha.

7. The dual vector system according to any one of items 1 to 6, wherein the first viral vector further comprises a gene sequence encoding a Cas9 or Cpf1 nuclease linked to one or two NLS.

8. The dual vector system according to any one of items 1 to 7, wherein the target gene sequence suppresses a gene product essential for infectious baculovirus virion generation, specifically selected from the group consisting of vp80, vp39, vp1054, gp64, p74, p24, vp1054, lef-1, lef-2, lef-4, lef-9, lef-11, pk1, vlf-1, bv-c42, bv-c27, Ac9, Ac25, Ac51, Ac53, Ac73, Ac75, Ac76, Ac78, Ac79, Ac81, Ac81, Ac82, Ac83, Ac92, Ac106/107, Ac109, Ac132, Ac146, 38K, and p6.9.

9. The dual vector system according to any one of items 1 to 8, wherein the promoter linked to the T7 polymerase is an early viral promoter, specifically selected from iE1, pe38, me53, lef3, gp64, vp39, and he65 promoter or a cellular promoter.

10. The dual vector system according to any one of items 1 to 9, wherein the viral vector, specifically the baculovirus vector is derived from nuclear polyhedrosis virus (NPV), specifically from Autographa californica multiple nuclear polyhedrosis virus (AcMNPV).

11. A recombinant insect or other animal cell line comprising the vector systems according to any one of items 1 to 10.

12. The insect cell line according to item 11, wherein said cell line is derived from Spodoptera frugiperda, Trichoplusia ni, Bombyx mori, Plutella sylostella, Manduca sexta and Mamestra brassicae, Helicoverpa armigera, Antheraea pernyi, Culex nigripalpus, Drosophila melanogaster, specifically selected from the group of Sf9, Sf21, Tnao38, High Five™, and Mimic™ cells.

13. A method for production of a recombinant protein in cells of animal origin, specifically of insect or mammalian origin, comprising the steps of:
introducing the vector system according to any one of items 1 to 12 into animal cells, specifically insect or mammalian cells,
cultivating the cells under conditions allowing expression of the recombinant protein and simultaneous downregulation of expression of the target protein, and

isolating the recombinant protein.

14. A method for production of a recombinant protein in cells of insect origin, comprising the steps of:
introducing the baculovirus vector system according to any one of items 3 to 12 into insect cells,
cultivating the cells under conditions allowing expression of the recombinant protein and simultaneous downregulation of expression of the target protein, and
isolating the recombinant protein.

15. A method for production of a recombinant protein in cells of animal origin, specifically of insect or mammalian origin comprising the steps of:
introducing the first viral vector of the dual vector system according to any one of items 1 to 12 comprising a gene sequence encoding a recombinant protein into animal, specifically insect or mammalian cells,
cultivating said cells under conditions allowing virus propagation and expression of the recombinant protein,
introducing the second viral vector of the dual vector system according to any one of items 1 to 12 into said cells,
cultivating the cells under conditions wherein expression of the target protein is downregulated, and isolating the recombinant protein.

16. A method for production of a recombinant protein in cells of insect origin comprising the steps of:
introducing the first baculovirus vector of the dual vector system according to any one of items 3 to 12 comprising a gene sequence encoding a recombinant protein into insect cells,
cultivating said cells under conditions allowing virus propagation and expression of the recombinant protein,
introducing the second baculovirus vector of the dual vector system according to any one of items 3 to 12 into said cells,
cultivating the cells under conditions wherein expression of the target protein is downregulated, and
isolating the recombinant protein.

17. The method according to any one of items 13 to 16, wherein expression of the target protein is downregulated due to the presence of effective amounts of ncRNA specifically targeting mRNA encoding said target protein.

18. The method according to item 17, wherein expression of the target protein is downregulated due to functional knock-out of the target gene sequence using gRNA-programmable Cas9 or Cpf1 nucleases.

The foregoing description will be more fully understood with reference to the following examples. Such examples are, however, merely representative of methods of practicing one or more embodiments of the present invention and should not be read as limiting the scope of invention.

### EXAMPLES

### Example 1

Development of an Artificial miRNA-Based, Inducible Knockdown System in Insect Cells

Summary

An inducible silencing system was established by engineering the natural RNA interference (RNAi) mechanism present in insect cells. RNAi is a conserved biological process using short RNA molecules, the small interfering RNAs (siRNAs), to sequence-specifically destroy target mRNAs and thus regulate gene expression (Hannon, 2002). SiRNAs are ˜20-25-nucleotide (nt) long non-coding double-stranded RNA molecules that are produced in response to foreign nucleic acid originating from exogenous invaders, such as viruses or transposons (Dana et al., 2017). Several studies have been carried out in insect cells focusing on the exploitation of the RNAi pathway to create a tool for gene function studies and to improve the production system by specific regulation of host or baculoviral gene expression (Huang et al., 2007; Kim et al., 2012; Zhang et al., 2018). A possible setup is based on the fact that siRNAs targeting a chosen gene can be embedded within endogenous primary microRNA (pri-miRNA) transcripts that serve as backbones (Haley et al., 2008; Zhang et al., 2014). Thus, the pri-miRNA carrying the synthetic siRNA, or so-called artificial microRNA (amiRNA), mimics the natural transcript and is recognized and processed by the host cell's microRNA biogenesis pathway (Bofill-De Ros and Gu, 2016).

The pri-miRNA transcript of Autographa californica multiple nuclear polyhedrosis virus miR-1 (AcMNPV-pri-miR-1) is the first and to our knowledge, the so far only miRNA discovered in the genome of AcMNPV, originally regulating the expression of the viral gene ODV-E25 (Zhu et al., 2013). To reveal and assess its silencing capabilities as a pri-miRNA mimic backbone, the original siRNA duplex within the stem-loop structure was replaced by a synthetic sequence targeting the enhanced yellow fluorescent protein (eYFP), which herein served as model for targeting a protein essential for virus production. Additionally, four different structural versions of the natural transcript were created by applying small changes in the stem sequences and the flanking regions according to general design rules (Bofill-De Ros and Gu, 2016). To evaluate the silencing potency of these customized constructs, a plasmid-based screening was carried out. According to this data, the most effective stem-loop structure was selected for insertion into the genome of AcMNPV. For the transcriptional control of the most effective silencer mimic, a T7-based expression system was developed. The bacteriophage T7 transcription machinery is an attractive system due to its strict promoter selectivity and high catalytic activity (Chamberlin et al., 1970). Several successful attempts have been made in the past years with the aim of utilizing this 100 kDa prokaryotic enzyme for protein expression in eukaryotic cells, including mammalian (Lieber et al., 1989), insect (Polkinghorne and Roy, 1995; van Poelwijk et al., 1995) and plant (Nguyen et al., 2004) cell lines.

The experimental setup presented here relies on two viral vectors. One recombinant baculovirus was designed to harbor an amiRNA construct linked to the bacteriophage T7 promoter, while the corresponding T7 RNA polymerase (T7 RNAP) with an additional nuclear localization signal is expressed by a second viral vector. The selective transcriptional activity, together with the fact that the functional expression of the amiRNA construct depends on the presence of the T7 RNAP that is encoded on a separate virus form the basis of the inducible system. Even when essential genes are targeted for downregulation, the two viral expression vectors can be produced efficiently whenever they are separate from each other. We could show that the T7 RNAP is functional in Sf9 cells as it activates transcription of genes that are under control of the T7 promoter. We further demonstrated that amiRNAs are functional after transcription by the T7 RNAP as they successfully downregulated the reporter gene eYFP. Our T7-based inducible expression system may serve as a valuable tool for gene regulation during a production process, e.g. by altering the glycosylation pattern or downregulating essential genes such as proteases or proteins involved in baculovirus assembly.

Materials and Methods

Insect Cells and Culture Conditions

Sf9 cells (ATCC CRL-1711) were propagated in HyClone SFM4 insect cell medium (GE Healthcare, Little Chalfont, UK) supplemented with 0.1% Pluronic F68 (Sigma-Aldrich, St. Louis, Mo., USA). 50 ml suspension cultures were cultivated in 500 ml flasks at 27° C. with a shaker speed of 100 rpm.

amiRNA Plasmid Constructs

The 600 nucleotide (nt) long promoter sequence upstream of the baculoviral immediate-early gene (ie1) was PCR amplified using the baculovirus shuttle vector originating from Max Efficiency DH10Bac cells (Invitrogen, Carlsbad, Calif., USA) as template. The fragment was then cloned between the ClaI and BamHI restriction sites of the MultiBac acceptor vector pACEBac1 (Geneva Biotech), thereby replacing the original polyhedrin (polh) promoter and resulting in the pACEBac1ie1 vector. The baculovirus shuttle vector harbored by DH10MultiBacY cells (Geneva Biotech) was used as template to obtain a PCR fragment of the gene encoding the eYFP (KT878739), which was cloned into the pACEBac1ie1 vector and thus gave rise to the pACEBac1ie1eYFP reporter plasmid.

To facilitate the setup of the inducible system, a special donor vector was created. A 344 nt long fragment containing (in order) the T7 RNAP promoter sequence, 120 nt-s of the 5′ flanking region of the natural miR-1 precursor hairpin (pre-miR-1) (Zhu et al., 2013) a mini multi cloning site (MCS), 120 nt-s of the 3′ flanking region of the pre-miR-1 hairpin and the TCD terminator sequence was chemically synthetized by IDT (Leuven, Belgium). After PCR amplification, the product was cloned between the SpeI and PmeI sites of the MultiBac donor vector pIDS (Geneva Biotech), thus replacing the original cloning cassette and resulting in the pIDST7amiR plasmid. The sequence of the above-described T7amiR fragment embedded in pIDS is presented in Table 1.

TABLE 1
Sequences used for the cloning of the artificial miRNA constructs.

| Fragment | |
|---|---|
| name | Nucleotide sequence 5′ to 3′ |
| T7amiR | TAATACGACTCACTATAGGGCTGCAGGTCTATAGATAGCGGTTTTT |
| fragment | CGGCAATATACACTTGGCTCAATTTATTATCGCCGTGTGCGATGCG |
| | CAAGTTGGCCACCCGGCCGTTATTCAGCTTTACGTTTAATTGTTTGT |
| | TCTCGTC |
| | ggatccgaattcctcgagtctagaAAATTTAATGCATTCGTCCAATAAAGATAAA |
| | ACAGTATGAGCAAAACGATAAGTAACACGATTCCCCACATGATTTG |
| | TTTTAATTTACAATTTCAATTCCAATGAGATTTAGGTTGTGCAGGTAC |
| | CCTAGCATAACCCCTTGGGGCCTCTAAACGGGTCTTGAGGGGTTTT |
| | TTG (SEQ ID NO. 1) |
| amiR-1A | TCAGCTTTACGTTTAATTGTTTGTTCTCGTCTAAGGCTACGTCTATA |
| stem loop | CTGCTCTATCCTAAACTGGATGATATAGACGTTGTGGCCTTGAAATT |
| | TAATGCATTCGTCCAATAAAGATAAA (SEQ ID NO. 2) |
| amiR-1As | TCAGCTTTACGTTTAATTGTTTGTTCTCGTCTAAACACTCTCAGTAA |
| stem loop | CTGCGACTCCCTAAACTGGGATCTTACTGAGACAGGTGTTTGAAAT |
| | TTAATGCATTCGTCCAATAAAGATAAA (SEQ ID NO. 3) |
| amiR-1B | ggatccTAAGGCCACAACGTCTATATCATCCTAAACTGGATGATATAG |
| stem loop | ACGTTGTGGCCTTAtctaga ((SEQ ID NO. 4) |
| amiR-1Bs | ggatccTAAACACTCTCTCGGGTAAAATCCCTAAACTGGGATTTTACC |
| stem loop | CGAGAGAGTGTTTAtctaga (SEQ ID NO. 5) |
| amiR-1C | ggatccTAACAGCCACAACGTCTATATCATGCCTAAACTGGCATGATA |
| stem loop | TAGACGTTGTGGCTGTTAtctaga (SEQ ID NO. 6) |
| amiR-1Cs | ggatccTAAACACCTCTCTCAGGTAAAATCGCCTAAACTGGCGATTTT |
| stem loop | ACCTGAGAGAGGTGTTTAtctaga (SEQ ID NO. 7) |
| amiR-1D | ggatccTGAGCGTAACAGCCACAACGTCTATATCATGCCTAAACTGGC |
| stem loop | ATGATATAGACGTTGTGGCTGTTACATTCAtctaga (SEQ ID NO. 8) |
| amiR-1Ds | ggatccTGAGCGTAAACACCTCTCTCAGGTAAAATCGCCTAAACTGGC |
| stem loop | GATTTTACCTGAGAGAGGTGTTTACATTCAtctaga (SEQ ID NO. 9) |

The restriction enzyme sites are in italic small caps. The guide strands of the synthetic siRNA duplexes embedded in the amiRNA hairpins are underlined.

As a basis for the amiRNA constructs, the AcMNPV-pri-miR-1 transcript served as a backbone (Zhu et al., 2013). The original siRNA duplex within the transcript was replaced with a synthetic one containing a siRNA sequence previously proven to be highly effective against its original target the enhanced green fluorescent protein (eGFP) (Ui-Tei et al., 2004). Furthermore, small changes were applied to the stem sequences, to create in overall four altered versions of the AcMNPV-miR-1 hairpin structure: amiR-1A, amiR-1B, amiR-1C, amiR-1D (amiR-1A-D). In addition to the diversity in the stem-loop structures, there are differences in the length of the flanking regions as well that were obtained from the natural AcMNPV-pri-miR-1 transcript. Moreover, for each of the amiRNA constructs, a corresponding control was also created by scrambling up the sequence of the given eGFP targeting siRNA duplex incorporated in the amiRNA backbone: amiR-1As, amiR-1Bs, amiR-1Cs, amiR-1Ds (amiR-1As-Ds). Sequences of the modified amiRNA hairpin constructs and the scrambled controls are listed in Table 1.

The diverse design of the amiRNA constructs necessitated different cloning procedures for the structures. For amiR-1 B, amiR-1C, amiR-1 D (amiR-1B-D) and amiR-1Bs, amiR-1Cs, amiR-1Ds (amiR-1Bs-Ds), a method described previously (Haley et al., 2008) was applied. Briefly, each of the stem-loop structures were ordered as two single stranded, complementary, synthetic oligonucleotides (oligos) from Sigma-Aldrich (St. Louis, Mo., USA). The oligos were then pairwise annealed according to the manufacturer's instructions and subsequently cloned between the BamHI and XbaI sites of the mini MCS in between the 120 nt long flanking regions of the pre-miR-1 hairpin in the donor vector pIDST7amiR, thus resulting in the plasmids pIDST7amiR-1B-D and pIDST7amiR-1Bs-Ds. Furthermore, for the transfection experiments carried out in Sf9 cells that served as a preliminary screening of the constructs (see section “Screening of amiRNA constructs”), the backbone of the pACEBac1ie1 vector was used. The reason behind is that the baculoviral ie1 promoter is active upon transfection in insect cells. To this end, the plasmids pIDST7amiR-1B-D and pIDST7amiR-1 Bs-Ds served as template for the PCR amplification of the 6 different fragments, each containing the 120 nt 5′ flank, the stem-loop and the 120 nt 3′ flank (without the T7 RNAP promoter and terminator sequences). The amplified products were subsequently cloned between the SalI and NotI sites of pACEBac1ie1, giving rise to the plasmids pACEBac1ie1amiR-1B-D and pACEBac1ie1amiR-1 Bs-Ds.

The nucleotide sequences encoding the stem-loops amiR-1A and amiR-1As, including the 31 nt long flanking regions of the natural AcMNPV-pri-miR-1 transcript on both sides of the stems, were chemically synthetized as single pieces by IDT (Leuven, Belgium). For the screening experiments, the fragments were cloned—after PCR amplification—between the BamHI and EcoRI sites of the pACEBac1ie1 vector to create the pACEBac1ie1amiR-1A and pACEBac1ie1amiR-1As plasmids, respectively. Furthermore, for the setup of the inducible system, the pIDST7amiR backbone was used and following another PCR amplification, the resulting stem-loop fragments were cloned between the T7 RNAP promoter and terminator sequences (PstI and KpnI sites) of the donor plasmid. This removed the 120 nt 5′ flank, the mini MCS and the 120 nt 3′ flank necessary for the insertion of the annealed oligos and resulted in the vectors pIDST7amiR-1A and pIDST7amiR-1As. All of the plasmids described here were confirmed with sequencing.

Screening of amiRNA Constructs

The preliminary screening experiments for the estimation of the silencing effectiveness of the different stem-loop constructs comprised of the transfection of insect cells followed by visual estimation by fluorescence microscopy and subsequent flow cytometry analysis. Sf9 cells were seeded to 6-well plates with a density of 9×10⁵ cells/well and were then pairwise co-transfected with 200 ng of the reporter plasmid pACEBac1ie1-eYFP in combination with 2 μg of one of the eight following plasmids: pACEBac1ie1amiR-1A-D or pACEBac1ie1amiR-1As-Ds. The co-transfections were done with FuGene HD transfection reagent (Promega, Madison, Wis., USA) according to the manufacturer's instructions. 48 hours post-transfection (h p.t.), the eYFP fluorescence intensity was first evaluated using a Leica DM IL LED Inverted Laboratory Microscope and the Leica Application Suite v4.6 software (Leica Microsystems, Wetzlar, Germany). After harvesting the cells, flow cytometry analysis was carried out using a Gallios Flow Cytometer (Beckman Coulter, Vienna, Austria). For the evaluation of the raw data, the Kaluza1.2 software (Beckman Coulter, Vienna, Austria) was applied. All co-transfection experiments were repeated thrice.

Cloning, Detection and In Vitro Activity Assay of the Bacteriophage T7 RNA Polymerase

The T7 gene1 encoding the bacteriophage T7 RNAP (AM946981) was PCR amplified using the lambda DE3 prophage as template present in Escherichia coli BL21(DE3) cells (New England Biolabs, Ipswich, Mass., USA). To target the mature RNA polymerase into the nucleus of Sf9 cells, where it is needed for the generation of pri-miRNA transcript mimics harboring the amiRNAs, the forward primer used for the PCR amplification contained extra 36 nt encoding the SV40 T antigen nuclear location signal (Polkinghorne and Roy, 1995; van Poelwijk et al., 1995). The obtained fragment was cloned within the BamHI and XbaI sites of the pACEBac1ie1 vector, resulting in the pACEBac1ie1T7RNAP construct.

To generate the Ac-ie1T7RNAP recombinant AcMNPV, the pACEBac1ie1T7RNAP vector was transformed into Max Efficiency DH10Bac competent cells (Invitrogen, Carlsbad, Calif., USA). The purified bacmid DNA was then transfected into Sf9 cells with FuGene HD transfection reagent (Promega, Madison, Wis., USA) according to the manufacturer's instructions. The amplified viral stock of passage three was used to determine the viral titer by plaque assay.

For the detection of T7 RNAP by Western blot analysis and the subsequent activity assay, Sf9 cells cultivated in a 20 ml suspension culture were infected with the Ac-ie1T7RNAP recombinant baculovirus at an initial cell density of 1×10⁶ cells/ml with a multiplicity of infection (MOI)=5. Samples of 1×10⁶ cells taken at 2 days post-infection were used for subsequent SDS-PAGE and Western blot analysis, according to standard protocols (Duojiao et al., 2016). Briefly, for the protein separation by SDS-PAGE, a Novex™ 4-12% Tris-Glycine Mini Protein Gel (Invitrogen, Carlsbad, Calif., USA) was applied, followed by electroblotting onto a PVDF transfer membrane (GE Healthcare Life Sciences, Vienna, Austria). Immunodetection was carried out using a primary anti-T7 RNA polymerase mouse monoclonal antibody (US170566-3, Merck, Kenilworth, N.J., USA) in combination with a secondary anti-mouse IgG alkaline phosphatase labeled goat antibody (A5153-1ML, Merck, Kenilworth, N.J., USA). Subsequently, the results were evaluated by visual estimation after the development of the PVDF transfer membrane using BCIP/NBT (Promega, Madison, Wis., USA).

To prove the functionality of the recombinant T7 RNAP, in vitro RNA transcription reactions were set up (van Poelwijk et al., 1995) using the components of the HiScribe T7 Quick High Yield RNA Synthesis Kit (New England Biolabs, Ipswich, Mass., USA). In all three different reaction mixtures that were applied, a PCR fragment of 900 nt downstream of the T7 promoter was used as a template. The 20 μl positive control reaction contained 2 μl nuclease-free water, 10 μl NTP buffer mix, 1 μl of the template fragment (500 ng DNA), 5 μl (200 U) Murine RNase inhibitor (New England Biolabs, Ipswich, Mass., USA) and 2 μl of T7 RNA polymerase mix. Whereas the negative control and test reaction mixtures were composed of 10 μl NTP buffer mix, 1 μl of the template fragment (500 ng DNA) and 5 μl (200 U) Murine RNase inhibitor (New England Biolabs, Ipswich, Mass., USA). Additionally, the T7 RNA polymerase mix was exchanged to 4 μl of resuspended cell pellets. The cell lysates were prepared as follows: samples of 2×10⁶ cells originating from an uninfected and the Ac-ie1T7RNAP infected Sf9 cultures (see above) were centrifuged at 500×g for 10 min (Eppendorf microcentrifuge 5415R, Hamburg, Germany) to separate the cells from the supernatants. The pellets were then resuspended and homogenized in 20 μl PBS. For the negative control and test transcription reactions, 4 μl of the non-infected or Ac-ie1T7RNAP infected resuspended and homogenized cell pellets were used, respectively. After the transcription (2 h at 37° C.) and DNA-removal step (15 min at 37° C.), the final RNA products were visualized on a 1% agarose gel containing 1% v/v sodium hypochlorite for the inactivation of RNAses (Aranda P S et al., 2012).

Setup of the Inducible Knockdown System

Acceptor-donor fusion constructs were generated via Cre-LoxP recombination by merging the reporter plasmid pACEBac1ie1eYFP with either pIDST7amiR-1C or pIDST7amiR-1Cs, resulting in the T7amiR-1C_ie1eYFP and T7amiR-1Cs_ie1eYFP vectors, respectively. By transforming the fusions into Max Efficiency DH10Bac competent cells (Invitrogen, Carlsbad, Calif., USA) with FuGene HD transfection reagent (Promega, Madison, Wis., USA) according to the manufacturer's instructions, the Ac-T7amiR-1C_ie1eYFP and Ac-T7amiR-1Cs_ie1eYFP recombinant viruses were created. The titers of the amplified viral stocks of passage three were determined by plaque assay.

The silencing of eYFP using the inducible viral system was evaluated on the protein level with flow cytometry. To this end, Sf9 cells seeded into T25 flasks at a cell density of 2.5×10⁶ cells/flask were co-infected with Ac-ie1T7RNAP together with the Ac-T7amiR-1C_ie1eYFP or Ac-T7amiR-1Cs_ie1eYFP virus at various MOI combinations. 48 hours post-infection (h p.i.) the cells were harvested and the eYFP fluorescence intensity was measured with a Gallios Flow Cytometer (Beckman Coulter, Vienna, Austria). For the data analysis, the Kaluza1.2 software (Beckman Coulter, Vienna, Austria) was used. The co-infections were set up three times.

Detection of Mature amiRNAs

Co-infected Sf9 samples of the inducible viral system, originating from the cultures used for the flow cytometry experiments (see section “Setup of inducible knockdown system”), were also analyzed with regard to the expression of mature amiRNAs of the constructs amiR-1C and amiR-1Cs. As negative controls for amiR-1C and amiR-1Cs, Sf9 cells were seeded into T25 flasks at a cell density of 2.5×10⁶ cells/flask and were infected only with either the Ac-T7amiR-1C_ie1eYFP or the Ac-T7amiR-1Cs_ie1eYFP virus at MOI 5, respectively. Total RNA was extracted from 1×10⁶ infected cells at 48 h p.i. using TRIzol Reagent (Invitrogen, Carlsbad, Calif., USA) according to the manufacturer's instructions. Genomic DNA was removed with the TURBO DNA-free Kit (Invitrogen, Carlsbad, Calif., USA). 500 ng total RNA was reverse transcribed using specific stem-loop primers (Chen C et al., 2005) with the ProtoScript II First Strand cDNA Synthesis Kit (New England Biolabs, Ipswich, Mass., USA). The 25 μl end-point PCR reaction mixtures contained 1 μl cDNA as template, 5 μl OneTaq Standard Reaction Buffer (5×), 0.5 μl 10 mM dNTPs, 0.5 μl 10 μM forward primer, 0.5 μl 10 μM reverse primer, 0.125 μl OneTaq DNA Polymerase and 17.375 μl nuclease-free water. The PCR was run on a Piko 24 PCR machine (Thermo Fisher Scientific, Waltham, Mass., USA) according to the standard protocol recommended by the manufacturer for the OneTaq DNA Polymerase (New England Biolabs, Ipswich, Mass., USA). The PCR products were visualized on a 1.5% agarose gel.

Real-Time Quantitative PCR

Total RNA was extracted from 1×10⁶ cells with TRizol Reagent (Invitrogen, Carlsbad, Calif., USA) according to the manufacturer's instructions. The genomic DNA contamination of the RNA samples was removed with the TURBO DNA-free Kit (Invitrogen, Carlsbad, Calif., USA). Reverse transcription and subsequent real-time quantitative PCR (RT-qPCR) was carried out in a single reaction with the Luna Universal One-Step RT-qPCR Kit (New England Biolabs, Ipswich, Mass., USA) according to the manufacturer's instructions. For the quantification of eYFP mRNA levels, sequence specific primers were used (F: 5′-GGCACAAGCTGGAGTACAAC-3′, SEQ ID NO. 10; R: 5′-AGTTCACCTTGATGCCGTTC-3′, SEQ ID NO. 11) that were designed with the GenScript Real-time PCR Primer Design online software. As internal reference gene, the insect 28S rRNA (Chen et al., 2017) was applied (F: 5′-GCTTACAGAGACGAGGTTA-3′, SEQ ID NO. 28; R: 5′-TCACTTCTGGAATGGGTAG-3′, SEQ ID NO. 12). RT-qPCR was performed in 20 μl reactions consisting of 10 μl Luna Universal One-Step Reaction Mix (2×), 1 μl Luna WarmStart RT Enzyme Mix (20×), 0.8 μl Forward primer (10 μM), 0.8 μl Reverse primer (10 μM), 5 ng DNA-free total RNA template and nuclease-free water (fill up to 20 μl). The experiments were conducted on a BioRad C1000 Thermal Cycler in combination with a CFX96 Real-Time PCR Detection System (Hercules, Calif., USA) using the following program: reverse transcription at 55° C. for 10 min, initial denaturation at 95° C. for 1 min, 40 cycles of denaturation at 95° C. for 10 sec and extension at 60° C. for 30 sec (with plate read). Specific amplification was confirmed through melting curve analysis. Each co-infection experiment was repeated three times and the data was analyzed using the 2^{-ΔΔCq} method (Schmittgen and Livak, 2008). For the evaluation of the statistical significance, the Student's t-test was applied (P<0.05).

Results

Plasmid-Based Evaluation of the amiRNA Constructs

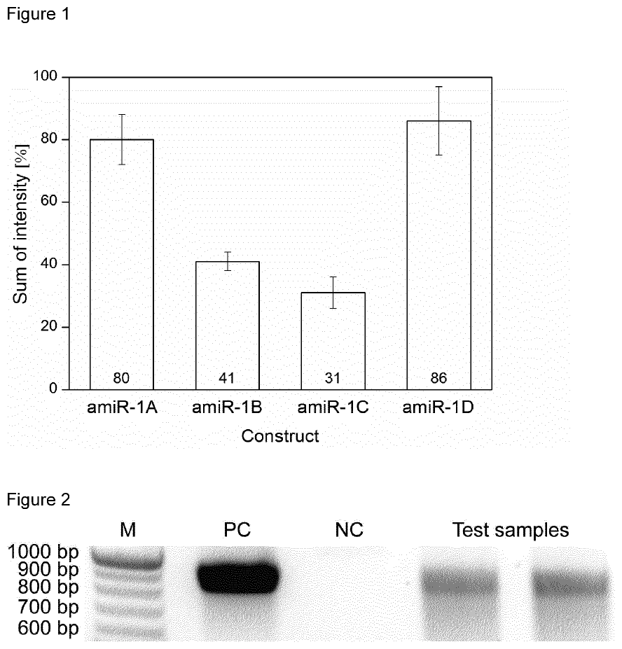

A plasmid-based screening assay was carried out, to evaluate and select the best amiRNA construct. Sf9 cells seeded into 6 well plates were transfected with 200 ng of the reporter plasmid pACEBac1ie1-eYFP in combination with 2 μg of one of the eight following plasmids: pACEBac1ie1amiR-1A-D or pACEBac1ie1amiR-1As-Ds (control). 48 h p.t. the silencing efficiency was evaluated by flow cytometry (FIG. 1). As a basis of comparison, the sum of intensity (overall fluorescence intensity) values were calculated by multiplying the fluorescent cell count with the arithmetic mean of the fluorescence intensity. Compared to the construct specific control transfections, the amiR-1B and amiR-1C hairpin constructs were the most efficient mimics in silencing the target gene eYFP with 59% and 69% reduction in the overall fluorescence, respectively. In contrast, the lowest efficiency of 14% reduction in eYFP intensity was achieved with amiR-1D, whereas the amiR-1A construct showed a minor reduction of 20% in eYFP intensity. Based on these results, the amiR-1C hairpin construct was considered most effective and was selected for further studies.

Expression of the Bacteriophage T7 RNA Polymerase

The T7 RNAP is a 100 kDa prokaryotic enzyme known for its tight promoter specificity and high catalytic activity (Studier and Moffatt, 1986). The fact that the T7 promoter does not occur in insect cells and is inactive in the absence of the T7 polymerase served as a basis for a two-vector-based inducible system. In order to test the functionality of the bacteriophage T7 RNAP in the insect cell system, we created the baculovirus Ac-ie1T7RNAP expressing the bacteriophage T7 RNAP with an additional nuclear location signal of the SV40 T antigen (van Poelwijk et al., 1995). Sf9 cells were infected with the Ac-ie1T7RNAP baculovirus, then samples (cell pellets) were collected 2 days post-infection. First, the cell pellets were used to confirm the expression of the T7 polymerase by Western blot analysis. Second, to assess the activity of the enzyme, an in vitro transcription assay (van Poelwijk et al., 1995) was carried out using a short DNA sequence downstream of the T7 promoter as a template. The transcription reactions were conducted using T7 RNAP of different sources. The positive control reaction (PC) contained a commercially available, purified RNAP mixture, whereas the negative control (NC) was set up with a few microliters of homogenized cell pellet from uninfected Sf9 cells. For the test reactions, the cell lysate of Sf9 cells infected with Ac-ie1T7RNAP was used. FIG. 2 shows the transcription products visualized on a 1% agarose gel. Notwithstanding that, the concentrated enzyme solution (PC) produced clearly higher amounts of template transcripts, the test reactions also contained the same size of transcripts indicating that the T7 RNAP produced in Sf9 cells was active. In contrast, no transcripts were detected in the negative control reaction.

Setup of the Inducible Knockdown System

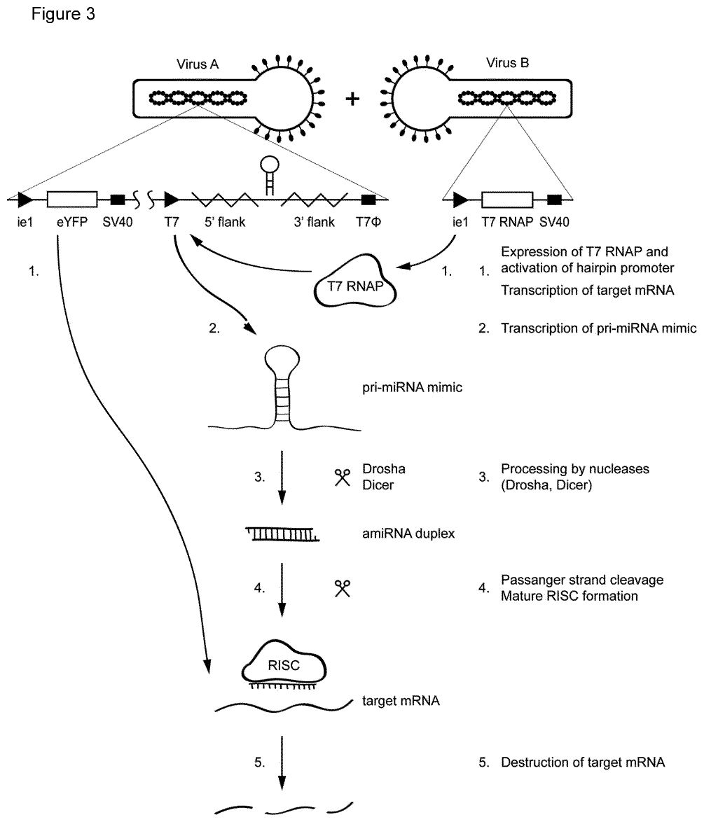

The amiR-1C hairpin structure was selected based on the preliminary screening experiments as the most efficient gene silencer. The baculoviruses Ac-T7amiR-1C_ie1eYFP and Ac-T7amiR-1Cs_ie1eYFP harboring the eYFP under the control of ie1 promoter, together with either the amiR-1C or the amiR-1Cs pri-miRNA under the control of T7 promoter, respectively. The inducibility of the system lies in the fact that in the absence of the T7 RNAP, the hairpin structures are not transcribed, as the T7 promoter is inactive without its corresponding T7 polymerase. Therefore, the previously evaluated Ac-ie1T7RNAP virus served as the inducer of the silencing effect. The mechanism of action is illustrated in FIG. 3. The main advantage of the system is that even genes that are essential for baculovirus propagation can be downregulated. Upon cultivating the two viruses separately, their infectious cycle and production process are undisturbed, but as soon as they are combined any desired silencing affect can be triggered.

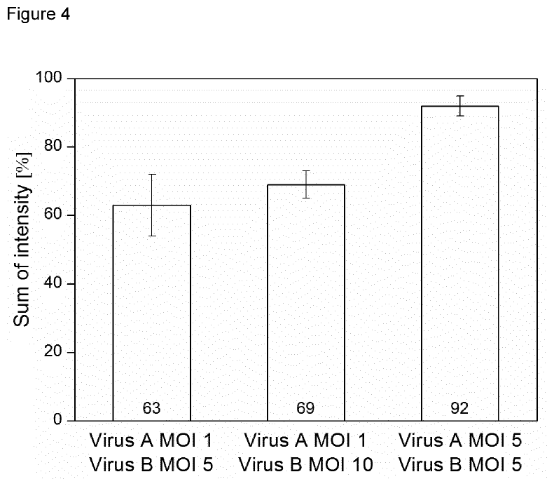

Sf9 cells were co-infected at various MOI combinations with the Ac-ie1T7RNAP virus and either the Ac-T7amiR-1C_ie1eYFP or the Ac-T7amiR-1Cs_ie1eYFP (control) baculovirus. Samples were collected 48 h p.i. to evaluate the silencing efficiency of the selected artificial hairpin structure (amiR-1C) in comparison to its sequence specific control (amiR-1Cs) on the protein level. FIG. 4 shows the flow cytometry results of co-infections, where the MOI of the Ac-T7amiR-1C_ie1eYFP and Ac-T7amiR-1Cs ie1eYFP (control) viruses were either 1 or 5, whereas the Ac-ie1T7RNAP was added at either MOI 5 or MOI 10 to the cultures. The 5-fold excess of the Ac-ie1T7RNAP virus resulted in a 37% decrease in the overall eYFP fluorescence intensity, whereas upon applying a 10-fold excess, a 31% reduction was observed. However, the combination of both viruses at MOI 5 turned out to be a less efficient setup, since only a minor reduction of 8% was perceivable in the fluorescence intensity. Nevertheless, the flow cytometry data on the protein level indicate the functionality of the inducible system on a viral basis, which was further confirmed on the RNA level with RT-qPCR.

Evaluation of the Inducible System on the RNA Level

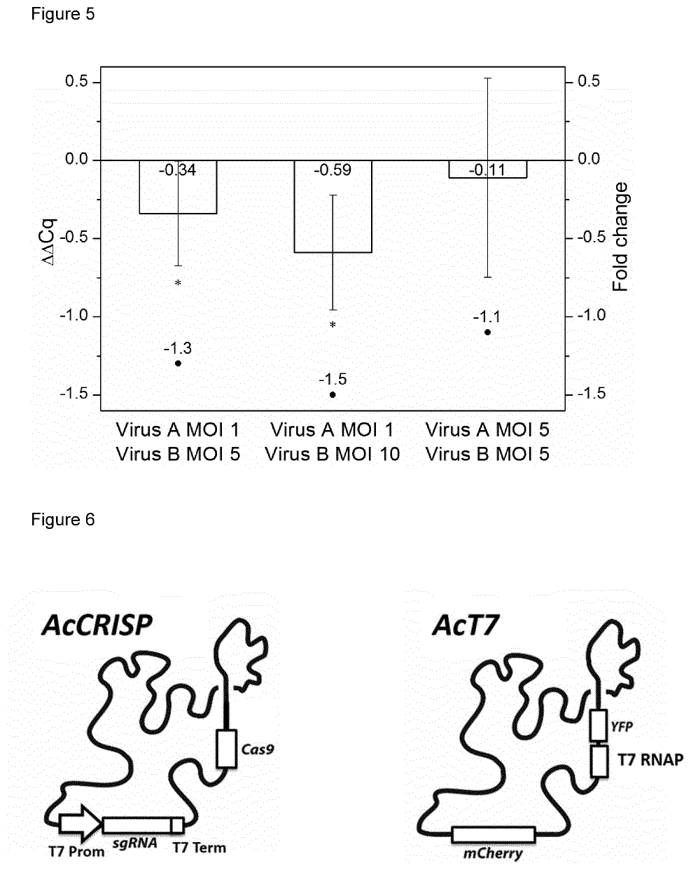

As a next step, silencing effects were investigated on the RNA level. To reveal, whether the reduction in overall eYFP fluorescence intensity was indeed the effect of specific downregulation, the decrease in the mRNA level was quantified by RT-qPCR. Samples were obtained from co-infected (Ac-T7amiR-1C_ie1eYFP or Ac-T7amiR-1Cs_ie1eYFP virus at MOI 1 or 5 in combination with Ac-ie1T7RNAP virus at MOI 5 or 10) Sf9 cultures. After total RNA extraction and genomic DNA removal, a one-step reverse transcription and RT-qPCR reaction was carried out using specific primers for eYFP. The data was evaluated using the 2^{-ΔΔCq} method (Schmittgen and Livak, 2008). For the calculation of ΔΔCq values and corresponding fold change values presented on FIG. 5, control values originating from co-infections with the construct specific control virus Ac-T7amiR-1Cs_ie1eYFP (ΔΔCq=0 or fold change of −1) under the same conditions were applied. The results were consistent with those from flow cytometry analysis. The co-infection with 5-fold excess (MOI 5) of the Ac-ie1T7RNAP virus compared to Ac-T7amiR-1C_ie1eYFP (MOI 1) resulted in a ΔΔCq value of −0.34 corresponding to a 1.3-fold reduction in the eYFP mRNA level, whereas applying the RNAP virus in 10-fold excess (MOI 10) lead to a stronger decrease with a ΔΔCq value of −0.59, indicating a 1.5-fold reduction in eYFP mRNA. However, the co-infection containing the same amounts of both viruses (MOI 5) did not result in a statistically significant suppression in the target mRNA level with a ΔΔCq value of −0.11 and a corresponding 1.1-fold reduction.

### Example 2

Introduction

A bacterial anti-viral adaptive defense system based on the RNA-guided nuclease Cas9 being used as DNA manipulation machinery heralded a transformative phase in the field of Biology and has been omnipresent in the scientific community and in the media during the last few years. The CRISPR (Clustered Regularly Interspaced Short Palindromic Repeats)-Cas9 system has been engineered into a simple and effective platform that allows for DNA manipulation at almost any position of a genome. It allows for creation of tailored changes in the DNA sequence including deletion, insertion, replacement, modification, labelling and transcriptional regulations (Hsu P. et al. (2014) Cell; 157(6): 1262-1278).

A knock-out set-up basically needs two components to be added to the DNA to be modified. The Cas9 nuclease and a gRNA designed by the customer, homologous for the DNA sequence to be modified. In vivo, the Cas9:gRNA complex binds to the DNA sequence to be modified and the Cas9 nuclease introduces a double-strand-break (DSB) at the specified gene or locus of interest. Highly conserved non-homologous end joining (NHEJ) DNA repair mechanisms repair the double-strand break in vivo, thereby creating small insertions or deletions (indels). This ultimately results in frame-shift mutations and destroys the open-reading frame of the target gene. Alternatively, when a DNA template with a desired mutation is supplied in vivo this may result in the substitution of the desired sequences at the site of the DSB by homology-directed repair (HDR) mechanisms (Hsu P., et al. (2014); Newman M. and Ausubel (2016).

Delivery of the CRISPR/Cas9 components—the Cas9 nuclease and gRNA—may be either in the format of (1) DNA encoding for the two components, (2) mRNA for Cas9 translation together with a separate gRNA or (3) a ribonucleoprotein complex consisting of recombinantly expressed Cas9 in complex with the gRNA (Newman M. and Ausubel (2016) Curr Protoc Mol Biol; 115: 31.4.1-31.4.6). As of its capability with large-scale bioprocesses in terms of scale-up and cost-effectiveness, the DNA format and a viral replication-competent delivery vehicle AcMNPV was chosen to ensure that most cells in a fermentation process receive the genome editing/knock-out message. The ultimate goal is the knock-out of essential genes for baculovirus budded virion generation during a bioprocess to reduce process-related impurities for later downstream processes of expression supernatants.

For in vivo gRNA synthesis, RNA polymerase II promoters, which constitute the vast majority of characterized promoters, are not applicable as of extensive RNA processing which ultimately leads to a physical separation of gRNA from the nuclease by export of RNA molecules into the cytoplasm. Therefore, in vivo generation of gRNAs is usually accomplished by using host endogenous RNA Polymerase III promoters of small nuclear RNAs such as the U6 and U3 snRNA promoters. Such promoters, however, are highly species-specific and have not yet been described for all biological systems. With the described system, we aim for the generation of a baculovirus-based CRISPR/Cas9 knock-out system that is characterized by its unprecedented versatility owing to its cross-species applicability. Generated Cas9-gRNA expression cassettes for viral or host genome engineering should be functional in all insect cell lines or cells of other animal origin amenable to baculovirus (AcMNPV) infection/transduction. Thereby, a single viral construct/backbone will be sufficient for being used in different insect cell lines, eliminating the need for preparing separate virus constructs with cell line-specific promoters (which are not yet available for all insect species) driving gRNA transcription. Therefore the gRNA was placed under control of the T7 RNA polymerase promoter followed by a T7 terminator. This should provide the CRISPR/Cas9 system with the desired flexibility and versatility.

For feasibility studies, a fluorescent reporter has been chosen as target gene that allows for easy evaluation of successful genome editing/gene knock-out by monitoring the mCherry phenotype of infected cells in comparison to controls. A dual vector system was generated consisting of an AcCRISP and AcT7 recombinant baculovirus vectors. The first vector, AcCRISP, carries the components of the CRISPR/Cas9 machinery—the Cas9 nuclease and gRNA. AcCRISP allows for in vivo expression of the Cas9 nuclease driven by the viral AcMNPV gp64 promoter. The promoter ensures that sufficient nuclease is available at the early and late phase of the infection cycle, before expression of the knock-out target mCherry (AcMNPV p6.9 promoter). The Cas9 coding sequence includes two nuclear-localisation signals that allow for retention of the nuclease in the nucleus. The second vector, AcT7, therefore encodes the T7 RNA polymerase controlled by the AcMNPV ie1 promoter along with the mCherry target gene expression cassette controlled by the AcMNPV p6.9 promoter and an AcMPNV vp39 promoter-driven YFP expression cassette for monitoring infection.

Methods

Cloning of the Dual Vector System AcCRISP and AcTARGET

A pBAC1gp64 transfer plasmid was generated by exchanging the polh promoter on the pBAC1 acceptor vector (Geneva Biotech) against the AcMNPV gp64 promoter. Briefly, pBAC1 was digested with ClaI and BamHI restriction enzymes and the gp64 promoter amplicon—amplified from an isolated MultiBac bacmid—was digested with the same enzymes and ligated into the prepared vector. The Cas9 coding sequence (codon-optimized for expression in Drosophila) was PCR-amplified from the plasmid pBS-Hsp70-Cas9 (Addgene), digested with EcoRI and HindIII and was cloned into pBAC1gp64 interjacent the gp64 promoter and SV40 terminator, yielding pBAC1gp64-Cas9. The Cas9 sequence includes an N-terminal 3× Flag-tag (5′-GACTATAAGGACCACGACGGAGACTACAAGGATCATGATATTGATTACAAAGACG ATGACGATAAG-′3, SEQ ID NO. 13) and SV40 nuclear localisation signal (5′-CCAAAGAAGAAGCGGAAGGTC-3′, SEQ ID NO. 14) and a C-terminal nucleoplasmin nuclear localisation signal (5′-AAAAGGCCGGCGGCCACGAAAAAGGCCGGCCAGGCAAAAAAGAAAAAG-3′, SEQ ID NO. 27).

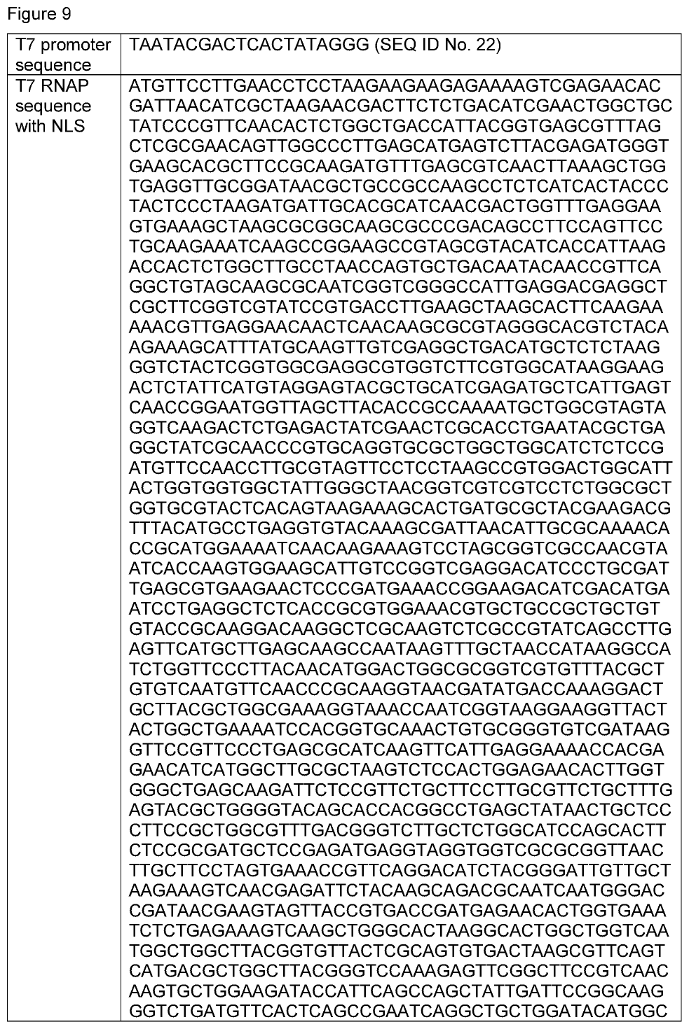

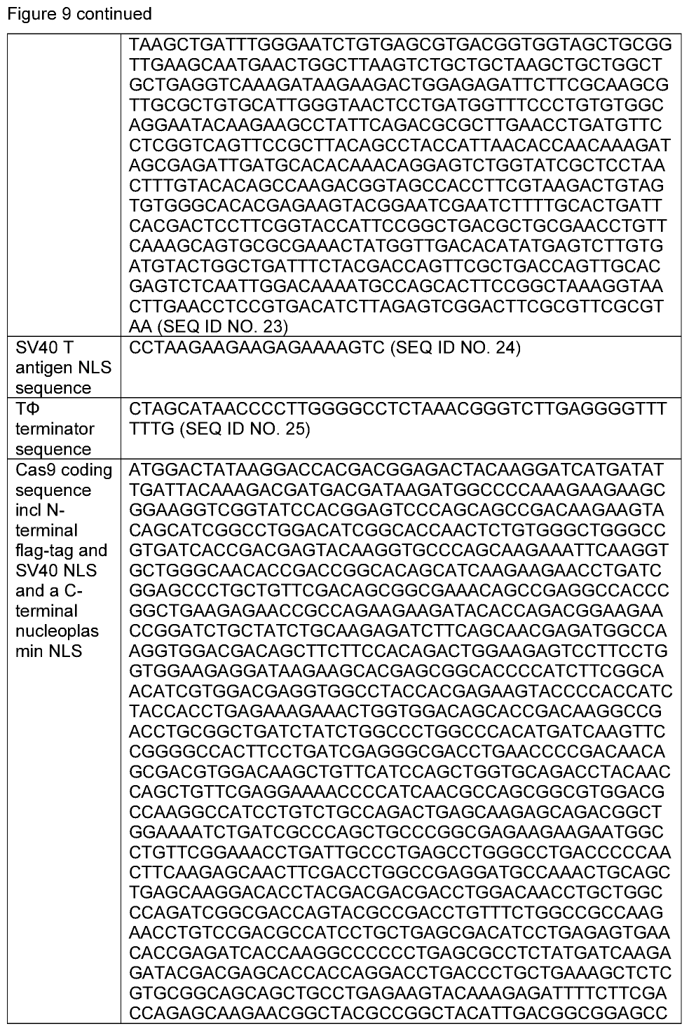

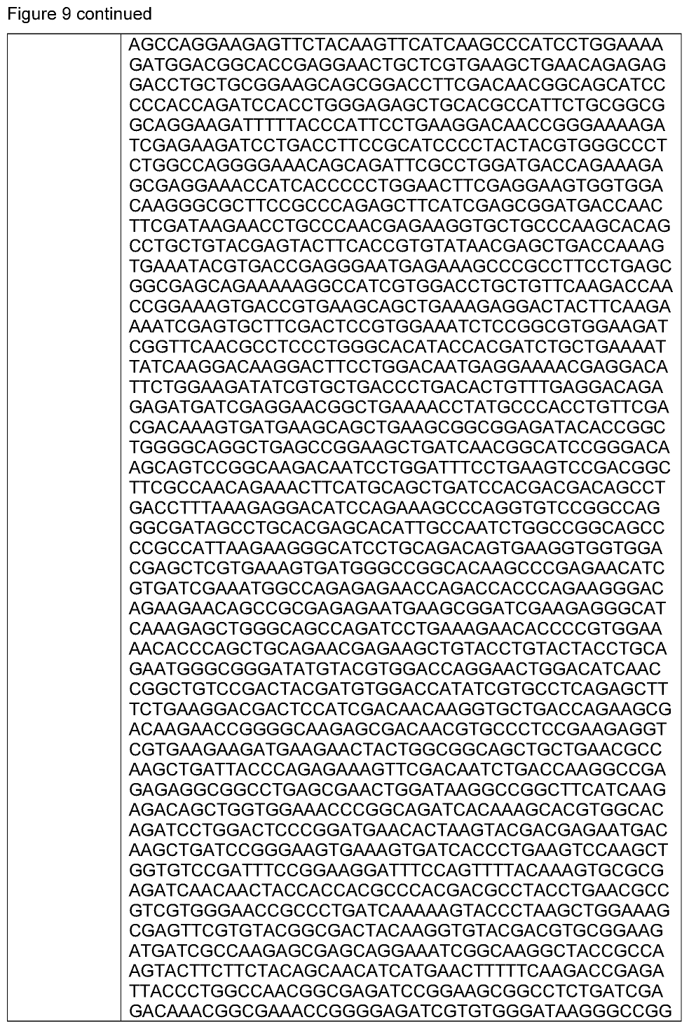

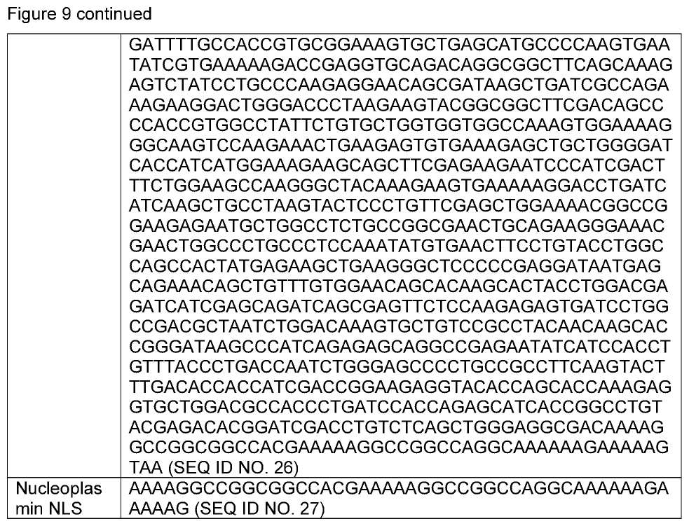

The polh promoter and SV40 terminator sequence from donor transfer vector pIDK (Geneva Biotech) were deleted using SpeI and PmeI restriction sites. gRNA amplicons harbouring a 5′ T7 promoter sequence and a 3′ T7 terminator sequence were generated (FIG. 9: SEQ ID No. 25) using Q5 polymerase (New England Biolabs) and were cloned into the modified pIDK vector, generating pIDKT7-gRNA1-T7 and pIDKT7-gRNA2-T7. Acceptor vector pBAC1gp64-Cas9 was fused with pIDKT7-gRNA1-T7 or pIDKT7-gRNA2-T7 using Cre recombinase-mediated recombination (New England Biolabs), resulting in plasmids pFus-gp64Cas9xT7gRNA1 and pFus-gp64Cas9xT7gRNA2. All constructs were analysed by restriction analysis and sequences were verified by sequencing. The fusion plasmids were transformed into DH10MultiBac (Geneva Biotech) and recombinant bacmids were isolated and transfected into Sf9 insect cells using the Fugene HD transfection reagent (Promega). Isolated virus seed stocks—termed AcCRISPmC1 and AcCRISPmC2—were amplified and viral working stocks titered using plaque assay.

A pBAC1p6.9 transfer plasmid was generated by exchanging the polh promoter on the pBAC1 acceptor vector against the AcMNPV p6.9 promoter. Briefly, pBAC1 was digested with ClaI and BamHI restriction enzymes and the p6.9 promoter, amplified from an isolated MultiBac bacmid—was digested with the same enzymes and ligated into the prepared vector, generating pBAC1p6.9.

For generation of the AcT7 virus, pACEBac1ie1T7RNAP was digested with SpeI and AvrII to cut out the whole T7RNAP expression cassette from the vector. The digested and purified expression cassette was cloned into the AvrII-digested vector pBAC1p6.9-mCherry to generate pBAC1p6.9-mCherry-ie1T7RNAP.Donor vector pIDC (Geneva Biotech) was digested with ClaI and BamHI for removing the polh promoter and exchange against the AcMNPV vp39 promoter amplified from the MultiBac bacmid, resulting in pIDCvp39. The fluorescence reporter eYFP was amplified from a bacmid isolated form EmBacY cells (Geneva Biotech) and was ligated into the pIDCvp39 vector at the BamHI and XbaI sites, resulting in pIDCvp39-eYFP. The fluorescence reporter was introduced into the loxP site of the MultiBac bacmid by transforming electrocompetent MultiBacCre cells with pIDCvp39-eYFP, yielding EmBacvp39Y cells. A positive transformant was used for the preparation of electrocompetent EmBacvp39Y cells that were used for the transformation pBAC1p6.9-mCherry-ie1T7RNAP to yield the recombinant bacmid AcTARGETmC. Recombinant bacmids were isolated and successful integration of the plasmid into the viral backbone was verified by blue-white screening and colony PCR. Recombinant bacmid was transfected into Sf9 insect cells using Fugene HD transfection reagent (Promega). The isolated virus seed stocks—termed AcTARGETmC—was amplified and viral working stocks tittered using plaque assay.

TABLE 2

| Fragment | |
|---|---|
| name | Nucleotide sequence 5′ to 3′ |
| T7P-gRNA | TAATACGACTCACTATAGGGGGTGTTATGAACTTCGAAGAGTTTT |
| 1-T7T | AGAGCTAGAAATAGCAAGTTAAAATAAGGCTAGTCCGTTATCAA |
| | CTTGAAAAAGTGGCACCGAGTCGGTGCCTAGCATAACCCCTTG |
| | GGGCCTCTAAACGGGTCTTGAGGGGTTTTTTG (SEQ ID NO. 16) |
| T7P-gRNA | TAATACGACTCACTATAGGGTGAAGGTCGTCCATACGAGTTTTA |
| 2-T7T | GAGCTAGAAATAGCAAGTTAAAATAAGGCTAGTCCGTTATCAAC |
| | TTGAAAAAGTGGCACCGAGTCGGTGCCTAGCATAACCCCTTGG |
| | GGCCTCTAAACGGGTCTTGAGGGGTTTTTTG (SEQ ID NO. 17) |

In Vitro Experiments

In Vitro Cas9 Cleavage Assay to Test Efficacy of Two Different T7RNAP-Synthesized sgRNAs

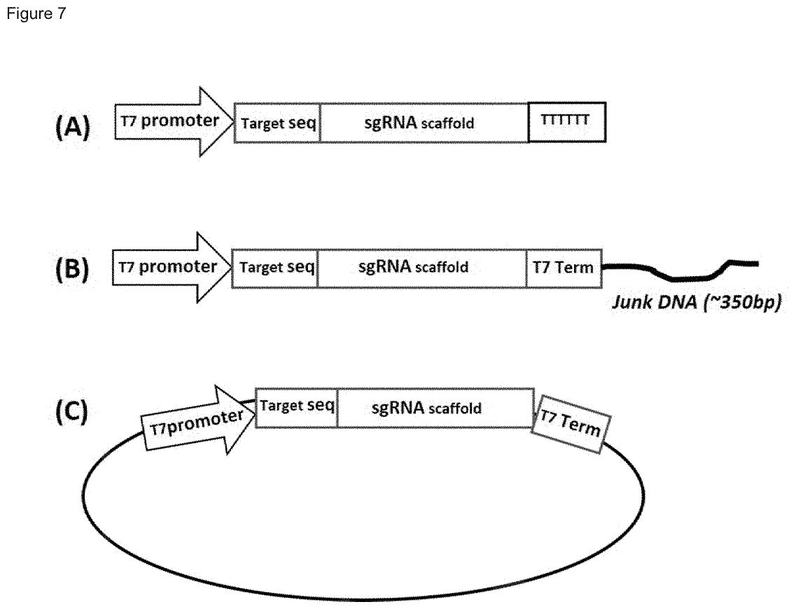

Single guide RNAs (sgRNAs) were transcribed in vitro with the HiScribe™ T7 Quick High Yield RNA Synthesis Kit (New England Biolabs) according to the manufacturer's protocol. Briefly, the transcription templates were prepared by PCR amplification using high-fidelity Q5 polymerase (New England Biolabs). Primers were designed to incorporate the minimal T7 promoter sequence (TAATACGACTCACTATA, SEQ ID No. 18) followed by three GGG bases for efficient transcription start initiation and a T-stretch termination signal (FIG. 7A)

Cas9-sgRNA complexes were allowed to assemble by incubating Cas9 nuclease and in vitro transcribed sgRNAs for 10 at 25° C. in Cas9 reaction buffer before addition of addition of substrate DNA. Cleavage assays were conducted in a reaction volume of 25 μL with 145 nM Cas9 nuclease from S. pyogenes (New England Biolabs), 145 nM sgRNA and 14.5 nM PCR product (10:10:1 ratio for optimal cleavage efficiency) for 1 hour at 37° C. The reaction was further incubated with 1 μL Proteinase K (20 mg/mL) at room temperature before fragment analysis on an agarose gel and sequencing.

Evaluation of T7 Termination Efficiency and Functionality of Generated sgRNAs

An in vitro assay was performed to test the T7 termination efficiency and functionality of the T7-RNA polymerase-transcribed sgRNAs. T7 termination efficiency was evaluated on basis of two different transcription templates, a linear PCR fragment (FIG. 7B) and a circular plasmid (pIDKT7-gRNA2T7), the latter one representing the DNA form present in vivo (FIG. 7C) Briefly, pIDKT7-gRNA2T7 served as template for the generation of a linear PCR fragment encoding the whole transcription cassette followed a ˜350 bp junk DNA sequence derived from the vector. The circular plasmid and linear DNA fragment then served as transcription templates for the synthesis of sgRNA. using the HiScribe™ T7 Quick High Yield RNA Synthesis Kit (New England Biolabs) as described above. Transcribed sgRNAs were visualized on an agarose gel and employed in the Cas9 cleavage assay.

In Vivo Experiments

Sf9, Trichoplusia ni, High Five and an Drosophila insect cell lines will be co-infected with the AcT7 virus virus harbouring the mCherry expression cassette, an YFP reporter and an expression cassette allowing for ie1-promoter-driven T7 RNAP expression and either AcCRISPmc1 or AcCRISPmc2 for delivery of the CRISPR/Cas9 components. Knock-out efficiency will be evaluated on basis of loss of the mCherry fluorescent phenotype.

In Vitro Cas9 Cleavage Assay to Test Efficacy of Two Different sgRNAs

T7-RNAP-in vitro-transcribed gRNA is routinely employed for confirming functionality of designed gRNAs in in vitro Cas9 cleavage assays. Here, a short DNA amplicon encoding the T7 promoter and gRNA sequence is generated and serves as template for the generation of gRNAs using purified, commercially available T7 RNA polymerases. In vitro-transcribed gRNA is then employed in the in vitro assay, together with commercially available Cas9 nuclease and a DNA target template (in our case: PCR amplicon of the mCherry coding sequence, which is used herein as model for a gene product essential for virus propagation).

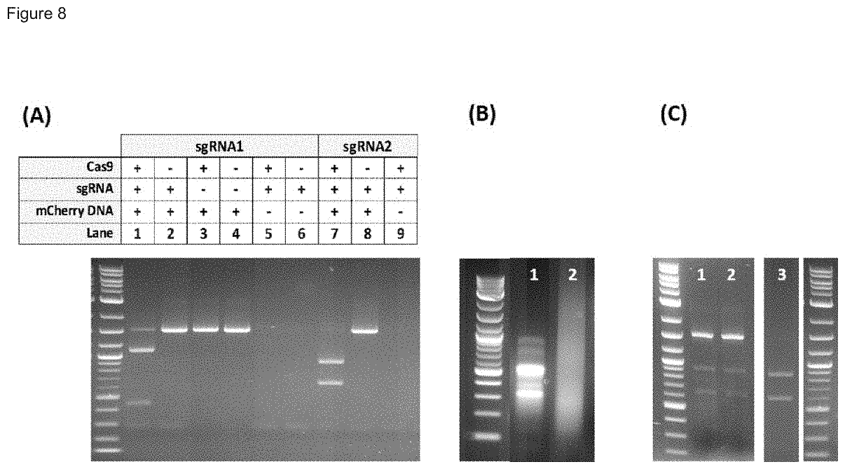

Two different T7 RNAP-transcribed sgRNAs targeting mCherry were tested using a Cas9 in vitro cleavage assay. As per the manufacturer's protocol, the template for sgRNA generation was a PCR product comprising the T7 terminator, mCherry target sequence, RNA scaffold followed by a short 7 bp T-stretch (FIG. 7A). A DNA template harbouring the mCherry coding sequence was mixed with a recombinant Cas9 and each sgRNA. The cleavage reactions were loaded onto an agarose gel and showed that both sgRNAs induce high cleavage efficiencies (sgRNA1: 91% (FIG. 8A, lane 1), sgRNA2: 98% (FIG. 8A, lane 7)). Control reactions lacked individual components of the cleavage assay, as given in the header. As the sequence of the DNA template for T7-RNAP-transcribed gRNA basically falls off after the gRNA sequence, we aimed to evaluate if the T7-RNAP produces functional gRNA in a setup mimicking an in vivo situation. In vivo, the gRNA is encoded on a plasmid/bacmid, rather than on a short linear DNA template. As the native T7 terminator is known to be prone to read-through transcription (Mairhofer J. et al., 2015, ACS Synthetic Biology 2015 4 (3), 265-273) it was tested in an in vitro set-up if the T7 system is capable of generating functional gRNA for Cas9-mediated knock-out. A linear PCR template encoding the transcription cassette followed by ˜350 bp vector-derived DNA (FIG. 7B) or a circular plasmid (FIG. 7C) encoding the transcription cassette were used as templates for in vitro RNA synthesis.

Synthesized sgRNAs were visualized on a 2% agarose gel. The linear transcription template yielded two prominent sgRNA species (FIG. 8A, lane 1), whereas the plasmid template resulted in the generation of a library of heterogenous sgRNAs of different lengths (FIG. 8B, lane 2). Although not providing full RNA synthesis termination efficiency, the generated sgRNAs do allow for Cas9-mediated DNA cleavage (FIG. 8B, lane 1: DNA template with junk DNA, lane 2: plasmid template). Even the very heterogenous sgRNA pool synthesized from the plasmid template (FIG. 8B, lane 2) supports equally efficient Cas9-mediated cleavage as compared to the more homogenous sgRNA pool generated from the PCR template including junk DNA (FIG. 8B, lane 1). This let us assume that T7RNAP polymerase generates functional sgRNA at sufficient levels to support Cas9-mediated DNA double-strand breaks even when synthesizing a heterogenic sgRNA pool resulting from T7 RNAP read-through events.

Cas9 cleavage is optimal when controlling the assay conditions as per the manufacturer's recommendations, by (1) using a short PCR template for sgRNA synthesis and (2) keeping a strict 10:10:1 molar ratio of the sgRNA:Cas9:DNA components. As these requirements were not fulfilled during assay conditions evaluating T7 termination efficiency (FIG. 8C, lane 1 and 2), it is obvious that cleavage efficiencies are lower than compared to the optimum assay conditions (FIG. 8C, lane 3).

### Example 3

The vector system is used for transduction, infection or transfection of mammalian and other animal cells, such as Hek, CHO, chicken cell lines or any cultivatable animal cell line. In this case the T7 RNAP and/or the Cas9 or Cpf1 nuclease must be expressed under a promoter that is recognized by the respective cell line, such as a AcMNPV ie1, SV40, CMV etc. The ncRNA or gRNA are expressed under the T7 RNAP promoter and terminated by the T7 or any other functional terminator.

### Example 4

The Constructs

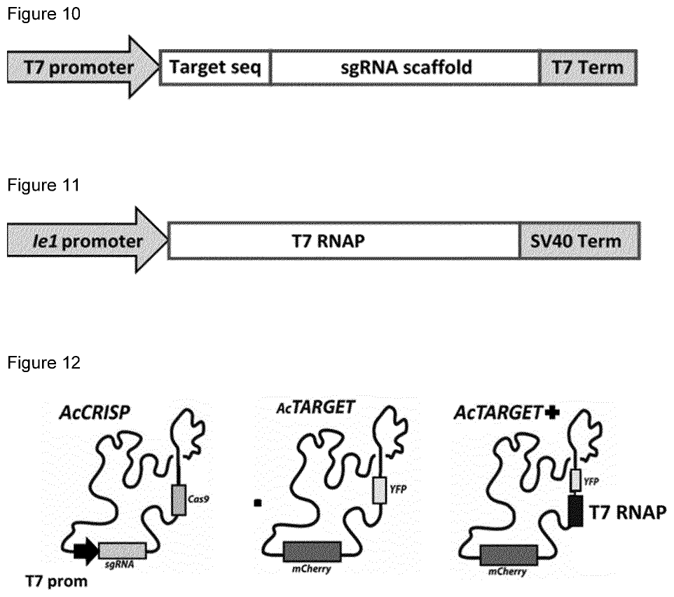

sgRNA construct for in vivo sgRNA transcription using the T7 promoter Two sgRNAs targeting the fluorescent reporter mCherry were designed using the online CRISPR design tool (https://benchling.com/crispr) and were cloned into the pIDK vector interjacent to the T7 promoter and terminator (FIG. 10). Upon co-expression of the already described T7 RNA-polymerase this construct allows for host cell-independent sgRNA transcription.

FIG. 10 shows a schematic picture of a sgRNA transcription cassette for in vivo sgRNA transcription.

T7 RNAP Vector Construct for In Vivo T7 RNA-Polymerase Expression in Insect Cells

The T7 RNA-Polymerase coding sequence (codon-optimized for expression in T. ni) was cloned into the pBAC1-p6.9-mCherry vector under control of the AcMNPV ie1 promoter and SV40 terminator (FIG. 11).

FIG. 11 provides a schematic picture of an T7RNAP expression cassette.

Generation of AcTARGET−/AcTARGET+ Viruses

Two different viral AcTARGET virus constructs were generated that allow for the easy evaluation of successful genome editing/gene knock-out by monitoring infected cells (YFP-positive) that show a loss of the mCherry-fluorescent phenotype. Both AcTARGET viruses harbour an AcMNPV vp39 promotor-driven YFP expression cassette for monitoring the infection status and the knock-out target mCherry under the AcMNPV p6.9 promoter. AcTARGET+ additionally harbours the abovementioned expression cassette for T7 RNAP expressed under the AcMNPV ie1 promoter, while AcTARGET− serves as control virus lacking T7RNAP. AcCRISP carries a Cas9 expression cassette using the AcMNPV gp64 promoter and the abovementioned T7-promoter-driven sgRNA cassette.

FIG. 12 shows the viral constructs.

Knock-Out Experiment

Co-infection experiments were performed in shaking flasks in a total volume of 10 mL with Sf9, Trichoplusia ni or High Five cells and Hyclone medium+0.01% (v/v) Pluronic-68. Cells were co-infected with AcCRISP and AcTARGET− or AcTARGET+ only at an MOI of 3 and 1 respectively and infected cells were investigated microscopically three days post infection.

Upon infection with both viruses, T7RNAP (encoded on virus AcTARGET+) transcribed the sgRNA specific for mCherry (encoded on virus AcCRISP) that forms a complex with Cas9 (encoded on virus AcCRISP) on the knock-out target DNA encoding for mCherry (encoded on virus AcTARGET+).

Results

Cells co-infected with AcCRISP and AcTARGET− display red fluorescence as evaluated by UV/Vis microscopy due to the higher abundance of mCherry protein available in the cytoplasm relative compared to YFP. In contrast, a successful knock-out upon co-infection with AcCRISP and AcTARGET+ should result in the loss of the red fluorescence phenotype due to the destruction of a functional mCherry gene by action of the Cas9, leading to a yellow/green fluorescence phenotype of cells as the YFP gene remains unaffected. One S. frugiperda (Sf9) and two T. ni cells lines were co-infected with above mentioned baculoviruses and the fluorescence phenotype was assessed by UV/VIS microscopy. While upon co-infection with AcCRISP and AcTARGET− all three cell lines showed a red/orange-fluorescence phenotype owing to the higher expression level of mCherry in contrast to YFP, co-infection with AcTARGET+ led to a strong reduction in mCherry expression as visualized by a drastic reduction in red-fluorescence phenotype and predominating yellow/green-fluorescence phenotype in the cell population.

### Example 5

Downregulation of Baculoviral Gp64

To downregulate the formation of baculoviral particles, baculoviral glycoprotein gp64, essential for efficient viral budding (Oomens and Blissard 1999), was subjected to knockdown. Therefore, amiR-1C was exchanged by an artificial miRNA which had incorporated instead of the siRNA duplex targeting eYFP, a siRNA duplex targeting gp64 (sequence taken from (Lee et al. 2015)) imbedded within the stem-loop framework of amiR-1C (gp64). Additionally, a specific control was generated, containing a scrambled version of the siRNA duplex within the same framework (gp64scr). Sequences used for cloning can be found in table 3.

TABLE 3

| Name | Nucleotide Sequence 5′ to 3′ |
|---|---|
| gp64 | ggatccTAAGCTGCGTGTCTGCTCATTAAAGCCTAAACTGGCTTTAATGA |
| | GCAGACACGCAGCTTAtctaga (SEQ ID No. 30) |
| gp64scr | ggatccTAAGTCATCGCATTGTCTGGACTAGCCTAAACTGGCTAGTCCA |
| | GACAATGCGATGACTTAtctaga (SEQ ID No. 31) |

Recombinant viruses were then produced as described before.

Sf9 cells were seeded in 25 cm² roux flasks to a density of 2.5×10⁶ cells/flask. Subsequently, they were co-infected with the virus providing the artificial miRNA precursor under control of the T7 promoter or the specific control virus at an MOI of 1 and the virus providing the T7RNA Polymerase at an MOI of 10. Samples were collected 48 h post-infection to evaluate the silencing efficiency of the artificial miRNA in comparison to its scrambled control, on the mRNA level. Total RNA was purified from 1×10⁶ cells with TRIzol Reagent (Invitrogen), genomic DNA was removed with the TURBO DNA-free Kit (Invitrogen) and downregulation was determined via RT-qPCR with the Luna Universal One-Step RT-qPCR Kit (New England Biolabs) according to the manufacturer's protocol. Gp64 mRNA quantification was carried out using gp64-specific primers (F: 5′-CGGCGTGAGTATGATTCTCAAA-3′ (SEQ ID No. 15), R: 5′-ATGAGCAGACACGCAGCTTTT-3′; SEQ ID No. 20). Insect cell 28S rRNA served as internal reference gene (F: 5′-GCTTACAGAGACGAGGTTA-3′ SEQ ID No. 28, R: 5′-TCACTTCTGGAATGGGTAG-3′, SEQ ID No. 21). Measurements were carried out with the MJ Mini™ cycler in combination with the MiniOpticon™ Real-Time PCR System with CFX Manager™ software (Bio-Rad, USA). Experiments were performed four times and data were analysed with the 2^{-ΔΔCq} method. To evaluate statistical significance a Student's t-test (p<0.05) was carried out.

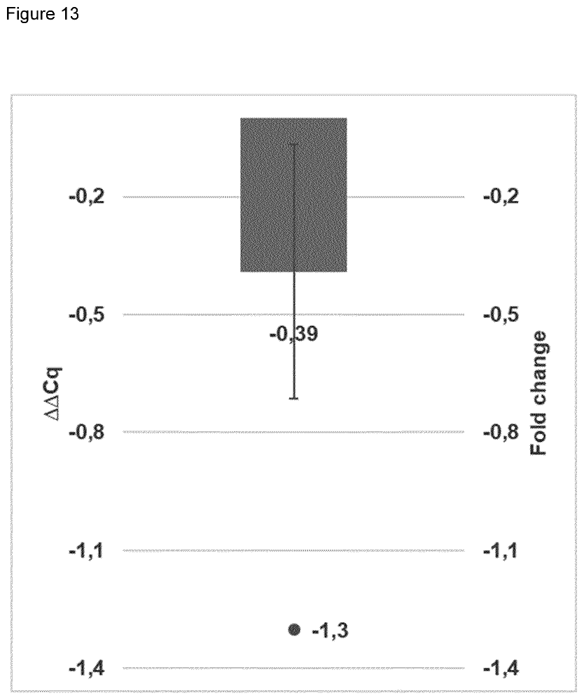

FIG. 13 shows the gp64-knock-down effect as evaluated by qPCR, i.e. the resulting ΔΔCq and corresponding Fold change value.

Targeting gp64 resulted in a ΔΔCq value of −0.39, indicating a 1.3 fold reduction of gp64 mRNA (see FIG. 13), confirming the functionality of the inducible knockdown system.

### REFERENCES

Boe, L., (1996) Estimation of plasmid loss rates in bacterial populations with a reference to the reproducibility of stability experiments. Plasmid 36, 161-167.
Bofill-De Ros, X., Gu, S., (2016) Guidelines for the optimal design of miRNA-based shRNAs. Methods 103, 157-166.
Chamberlin, M., McGrath, J., Waskell, L., (1970) New RNA Polymerase from Escherichia coli infected with Bacteriophage T7. Nature 228, 227.
Chen, Z.-S., Han, N.-N., Li, J.-H., Wan, H., Huang, G.-H., (2017) Selection and validation of reference genes for RT-qPCR analysis in Sf9 cell line infected by Heliothis virescens ascovirus 3h (HvAV-3h). Journal of Asia-Pacific Entomology 20, 463-468.
Dana, H., Chalbatani, G. M., Mahmoodzadeh, H., Karimloo, R., Rezaiean, O., Moradzadeh, A., Mehmandoost, N., Moazzen, F., Mazraeh, A., Marmari, V., Ebrahimi, M., Rashno, M. M., Abadi, S. J., Gharagouzlo, E., (2017) Molecular Mechanisms and Biological Functions of siRNA. International Journal of Biomedical Science: IJBS 13, 48-57.
Oomens, A. G., Blissard, G. W., (1999) Requirement for GP64 to drive efficient budding of Autographa californica multicapsid nucleopolyhedrovirus. Virology, 254, 297-314.
Duojiao, N., Peng, X., Sean, G., (2016) Immunoblotting and Immunodetection. Current Protocols in Immunology 114, 8.10.11-18.10.36.
Haley, B., Hendrix, D., Trang, V., Levine, M., (2008) A simplified miRNA-based gene silencing method for Drosophila melanogaster. Developmental biology 321, 482-490.
Hannon, G. J., (2002) RNA interference. Nature 418, 244.
Hsu, P. D., Lander, E. S., Zhang, F., (2014) Development and applications of CRISPR-Cas9 for genome engineering. Cell 157, 1262-1278.
Huang, Y., Deng, F., Hu, Z., Vlak, J. M., Wang, H., (2007) Baculovirus-mediated gene silencing in insect cells using intracellularly produced long double-stranded RNA. Journal of biotechnology 128, 226-236.
Jeng, S. T., Gardner, J. F., Gumport, R. I., (1992) Transcription termination in vitro by bacteriophage T7 RNA polymerase. The role of sequence elements within and surrounding a rho-independent transcription terminator. J Biol Chem 267, 19306-19312.
Kim, N.Y., Baek, J. Y., Choi, H. S., Chung, I. S., Shin, S., Lee, J. I., Choi, J. Y., Yang, J. M., (2012) Short-hairpin RNA-mediated gene expression interference in Trichoplusia ni cells. J Microbiol Biotechnol 22, 190-198.
Lee, H. S. et al., (2015) Small interfering (si) RNA mediated baculovirus replication reduction without affecting target gene expression, Virus Research, vol. 199, pp. 68-76
Lieber, A., Kiessling, U., Strauss, M., (1989) High level gene expression in mammalian cells by a nuclear T7-phase RNA polymerase. Nucleic acids research 17, 8485-8493.
Marek, M., van Oers, M. M., Devaraj, F. F., Vlak, J. M., Merten, O. W., (2011) Engineering of baculovirus vectors for the manufacture of virion-free biopharmaceuticals. Biotechnol Bioeng 108, 1056-1067.
Nguyen, H. T., Leelavathi, S., Reddy, V. S., (2004) Bacteriophage T7 RNA polymerase-directed, inducible and tissue-specific over-expression of foreign genes in transgenic plants. Plant biotechnology journal 2, 301-310.
Pijlman, G. P., van Schijndel, J. E., Vlak, J. M., (2003) Spontaneous excision of BAC vector sequences from bacmid-derived baculovirus expression vectors upon passage in insect cells. J Gen Virol 84, 2669-2678.
Polkinghorne, I., Roy, P., (1995) Transient expression in insect cells using a recombinant baculovirus synthesising bacteriophage T7 RNA polymerase. Nucleic acids research 23, 188-191.
Schmittgen, T. D., Livak, K. J., (2008) Analyzing real-time PCR data by the comparative CT method. Nature Protocols 3, 1101.
Studier, F. W., Moffatt, B. A., (1986) Use of bacteriophage T7 RNA polymerase to direct selective high-level expression of cloned genes. Journal of Molecular Biology 189, 113-130.
Steele, K. H. et al., (2017) Improving the baculovirus expression vector system with vankyrin-enhanced technology, Biotechnology Progress, vol. 33, no. 6, pp. 1496-1507
Ui-Tei, K., Naito, Y., Takahashi, F., Haraguchi, T., Ohki-Hamazaki, H., Juni, A., Ueda, R., Saigo, K., (2004) Guidelines for the selection of highly effective siRNA sequences for mammalian and chick RNA interference. Nucleic acids research 32, 936-948.
van Poelwijk, F., Broer, R., Belsham, G. J., Oudshoorn, P., Vlak, J. M., Goldbach, R. W., (1995) A hybrid baculovirus-bacteriophage T7 transient expression system. Bio/technology (Nature Publishing Company) 13, 261-264.
Van Poelwijk, F. et al., (1995) A Hybrid Baculovirus-Bacteriophage T7 Transient Expression System, Nature Biotechnology, vol. 13, no. 3, pp. 261-264
Zhang, J., He, Q., Zhang, C. D., Chen, X. Y., Chen, X. M., Dong, Z. Q., Li, N., Kuang, X. X., Cao, M. Y., Lu, C., Pan, M. H., (2014) Inhibition of BmNPV replication in silkworm cells using inducible and regulated artificial microRNA precursors targeting the essential viral gene lef-11. Antiviral research 104, 143-152.
Zhang, X., Xu, K., Ou, Y., Xu, X., Chen, H., (2018) Development of a baculovirus vector carrying a small hairpin RNA for suppression of sf-caspase-1 expression and improvement of recombinant protein production. BMC biotechnology 18, 24.
Zhu, M., Wang, J., Deng, R., Xiong, P., Liang, H., Wang, X., (2013) A MicroRNA Encoded by Autographa californica Nucleopolyhedrovirus Regulates Expression of Viral Gene ODV-E25. Journal of Virology 87, 13029-13034.

1. A dual vector system for production of one or more recombinant proteins of interest in insect cells, comprising:
a first baculovirus vector comprising a T7 promoter operably linked to a targeting sequence comprising a non-coding sequence followed by a T7 termination sequence, wherein expression of the targeting sequence can suppress one or more proteins essential for virus production,
a second baculovirus vector comprising a promoter operably linked to a T7 RNA polymerase encoding sequence, and
at least one gene sequence located on the first and/or second vector encoding one or more recombinant proteins of interest.

2. The dual vector system of claim 1, wherein the T7 RNA polymerase is linked to a nucleus localization sequence (NLS).

3. The dual vector system according to claim 1, wherein the expression product of the targeting sequence is an non-coding (nc) RNAi selected from the group consisting of guide RNAs (gRNA), endogenous primary-micro RNAs (pri-miRNA), artificial micro RNAs (amiRNA), small interfering RNAs (siRNA), short hairpin RNAs (shRNA), long hairpin RNAs (lhRNA), and polycystronic shRNAs.

4. The dual vector system according to claim 3, wherein the ncRNA encoded by the targeting sequence is further processed by one or more cellular enzymes.

5. The dual vector system according to claim 1, wherein the first baculovirus vector further comprises a gene sequence encoding a Cas9 or Cpf1 (Cas12a) nuclease linked to one or two NLS.

6. The dual vector system according to claim 1, wherein the target gene sequence suppresses a gene product essential for infectious baculovirus virion generation selected from the group consisting of vp80, vp39, vp1054, gp64, p74, p24, vp1054, lef-1, lef-2, lef-4, lef-9, lef-11, pk1, vlf-1, bv-c42, bv-c27, Ac9, Ac25, Ac51, Ac53, Ac73, Ac75, Ac76, Ac78, Ac79, Ac81, Ac81, Ac82, Ac83, Ac92, Ac106/107, Ac109, Ac132, Ac146, 38K, and p6.9.

7. The dual vector system according to claim 1, wherein the promoter operably linked to the T7 RNA polymerase encoding sequence is an early viral promoter.

8. The dual vector system according to claim 1, wherein the baculovirus vector is derived from a nuclear polyhedrosis virus (NPV).

9. A recombinant insect cell line comprising the dual vector system of claim 1.

10. The insect cell line according to claim 9, wherein said cell line is derived from a species selected from the group consisting of Spodoptera frugiperda, Trichoplusia ni, Bombyx mori, Plutella sylostella, Manduca sexta and Mamestra brassicae, Helicoverpa armigera, Antheraea pernyi, Culex nigripalpus, and Drosophila melanogaster.

11. A method for production of a recombinant protein in insect cells, comprising the steps of:
introducing the dual vector system according to claim 1 into insect cells,
cultivating the cells under conditions allowing expression of the recombinant protein and simultaneous downregulation of expression of the target protein, and
isolating the recombinant protein.

12. A The method for production of a recombinant protein in insect cells of claim 11, further comprising the steps of:
introducing the first viral vector of the dual vector system comprising a gene sequence encoding a recombinant protein into the insect cells,
cultivating said cells under conditions allowing virus propagation and expression of the recombinant protein,
introducing the second viral vector of the dual vector system into said cells, and
cultivating the cells under conditions wherein expression of the target protein is downregulated.

13. The method according to claim 12, wherein expression of the target protein is downregulated due to the presence of effective amounts of ncRNA specifically targeting mRNA encoding said target protein.

14. The method according to claim 12, wherein expression of the target protein is downregulated due to functional knock-out of the target gene sequence using gRNA-programmable Cas9 or Cpf1 (Cas12a) nucleases.

15. The dual vector system according to claim 4, wherein the one or more cellular enzymes are selected from the group consisting of Dicer and Drosha.

16. The dual vector system according to claim 7, wherein the promoter operably linked to the T7 RNA polymerase is a cellular promoter or a promoter selected from the group consisting of iE1, pe38, me53, lef3, gp64, vp39, and he65.

17. The dual vector system according to claim 8, wherein the nuclear polyhedrosis virus is Autographa californica multiple nuclear polyhedrosis virus (AcMNPV).

18. The insect cell line according to claim 10, wherein cells of the cell line are selected from the group consisting of Sf9, Sf21, Tnao38, High Five™, and Mimic™ Sf9 cells.


SEQUENCE LISTING (31 sequences)

SEQ ID NO: 1 - DNA, 344 bp, Artificial Sequence
  T7amiR fragment
taatacgactcactatagggctgcaggtctatagatagcggtttttcggcaatatacacttggctcaatttattatcgccgtgtgcgatgcgcaagttggccacccggccgttattcagctttacgtttaattgtttgttctcgtcggatccgaattcctcgagtctagaaaatttaatgcattcgtccaataaagataaaacagtatgagcaaaacgataagtaacacgattccccacatgatttgttttaatttacaatttcaattccaatgagatttaggttgtgcaggtaccctagcataaccccttggggcctctaaacgggtcttgaggggttttttg

SEQ ID NO: 2 - DNA, 120 bp, Artificial Sequence
  amiR-1A stem loop
tcagctttacgtttaattgtttgttctcgtctaaggctacgtctatactgctctatcctaaactggatgatatagacgttgtggccttgaaatttaatgcattcgtccaataaagataaa

SEQ ID NO: 3 - DNA, 120 bp, Artificial Sequence
  amiR-1As stem loop
tcagctttacgtttaattgtttgttctcgtctaaacactctcagtaactgcgactccctaaactgggatcttactgagacaggtgtttgaaatttaatgcattcgtccaataaagataaa

SEQ ID NO: 4 - DNA, 68 bp, Artificial Sequence
  amiR-1B stem loop
ggatcctaaggccacaacgtctatatcatcctaaactggatgatatagacgttgtggccttatctaga

SEQ ID NO: 5 - DNA, 68 bp, Artificial Sequence
  amiR-1Bs stem loop
ggatcctaaacactctctcgggtaaaatccctaaactgggattttacccgagagagtgtttatctaga

SEQ ID NO: 6 - DNA, 72 bp, Artificial Sequence
  amiR-1C stem loop
ggatcctaacagccacaacgtctatatcatgcctaaactggcatgatatagacgttgtggctgttatctaga

SEQ ID NO: 7 - DNA, 72 bp, Artificial Sequence
  amiR-1Cs stem loop
ggatcctaaacacctctctcaggtaaaatcgcctaaactggcgattttacctgagagaggtgtttatctaga

SEQ ID NO: 8 - DNA, 84 bp, Artificial Sequence
  amiR-1D stem loop
ggatcctgagcgtaacagccacaacgtctatatcatgcctaaactggcatgatatagacgttgtggctgttacattcatctaga

SEQ ID NO: 9 - DNA, 84 bp, Artificial Sequence
  amiR-1Ds stem loop
ggatcctgagcgtaaacacctctctcaggtaaaatcgcctaaactggcgattttacctgagagaggtgtttacattcatctaga

SEQ ID NO: 10 - DNA, 20 bp, Artificial Sequence
  primer
ggcacaagctggagtacaac

SEQ ID NO: 11 - DNA, 20 bp, Artificial Sequence
  primer
agttcaccttgatgccgttc

SEQ ID NO: 12 - DNA, 19 bp, Artificial Sequence
  primer
tcacttctggaatgggtag

SEQ ID NO: 13 - DNA, 66 bp, Artificial Sequence
  Cas9
gactataaggaccacgacggagactacaaggatcatgatattgattacaaagacgatgacgataag

SEQ ID NO: 14 - DNA, 21 bp, Sendai virus
ccaaagaagaagcggaaggtc

SEQ ID NO: 15 - DNA, 22 bp, Artificial Sequence
  primer
cggcgtgagtatgattctcaaa

SEQ ID NO: 16 - DNA, 164 bp, Artificial Sequence
  T7P-gRNA 1-T7T
taatacgactcactatagggggtgttatgaacttcgaagagttttagagctagaaatagcaagttaaaataaggctagtccgttatcaacttgaaaaagtggcaccgagtcggtgcctagcataaccccttggggcctctaaacgggtcttgaggggttttttg

SEQ ID NO: 17 - DNA, 162 bp, Artificial Sequence
  T7P-gRNA 2-T7T
taatacgactcactatagggtgaaggtcgtccatacgagttttagagctagaaatagcaagttaaaataaggctagtccgttatcaacttgaaaaagtggcaccgagtcggtgcctagcataaccccttggggcctctaaacgggtcttgaggggttttttg

SEQ ID NO: 18 - DNA, 17 bp, Artificial Sequence
  promoter
taatacgactcactata

SEQ ID NO: 19 - protein, 16 aa, Artificial Sequence
  NLS
KRPAATKKAGQAKKKK

SEQ ID NO: 20 - DNA, 21 bp, Artificial Sequence
  primer
atgagcagacacgcagctttt

SEQ ID NO: 21 - DNA, 19 bp, Artificial Sequence
  primer
tcacttctggaatgggtag

SEQ ID NO: 22 - DNA, 20 bp, Artificial Sequence
  T7 promoter sequence
taatacgactcactataggg

SEQ ID NO: 23 - DNA, 2688 bp, Artificial Sequence
  T7 RNAP sequence with NLS
atgttccttgaacctcctaagaagaagagaaaagtcgagaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 24 - DNA, 21 bp, Artificial Sequence
  SV40 T antigen NLS sequence
cctaagaagaagagaaaagtc

SEQ ID NO: 25 - DNA, 48 bp, Artificial Sequence
  terminator sequence
ctagcataaccccttggggcctctaaacgggtcttgaggggttttttg

SEQ ID NO: 26 - DNA, 4272 bp, Artificial Sequence
  Cas9
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 27 - DNA, 48 bp, Artificial Sequence
  Nucleoplasmin NLS
aaaaggccggcggccacgaaaaaggccggccaggcaaaaaagaaaaag

SEQ ID NO: 28 - DNA, 19 bp, Artificial Sequence
  primer
gcttacagagacgaggtta

SEQ ID NO: 29 - protein, 7 aa, Artificial Sequence
  NLS
PKKKRKV

SEQ ID NO: 30 - DNA, 72 bp, Artificial Sequence
  gp64
ggatcctaagctgcgtgtctgctcattaaagcctaaactggctttaatgagcagacacgcagcttatctaga

SEQ ID NO: 31 - DNA, 72 bp, Artificial Sequence
  gp64scr
ggatcctaagtcatcgcattgtctggactagcctaaactggctagtccagacaatgcgatgacttatctaga